SEQUENCE LISTING (66 sequences)

SEQ ID NO: 1 - protein, 278 aa, Artificial Sequence
  extracellular water-soluble domain of DLK1
ECFPACNPQNGFCEDDNVCRCQPGWQGPLCDQCVTSPGCLHGLCGEPGQCICTDGWDGELCDRDVRACSSAPCANNGTCVSLDDGLYECSCAPGYSGKDCQKKDGPCVINGSPCQHGGTCVDDEGRASHASCLCPPGFSGNFCEIVANSCTPNPCENDGVCTDIGGDFRCRCPAGFIDKTCSRPVTNCASSPCQNGGTCLQHTQVSYECLCKPEFTGLTCVKKRALSPQQVTRLPSGYGLAYRLTPGVHELPVQQPEHRILKVSMKELNKKTPLLTEG

SEQ ID NO: 2 - protein, 5 aa, Artificial Sequence
  CDR1 of heavy chain
SYAMN

SEQ ID NO: 3 - protein, 17 aa, Artificial Sequence
  CDR2 of heavy chain
TITATSGKTYYADSVKG

SEQ ID NO: 4 - protein, 14 aa, Artificial Sequence
  CDR3 of heavy chain
GESCSGGACSDFDY

SEQ ID NO: 5 - protein, 14 aa, Artificial Sequence
  CDR1 of light chain
TGTSSDIGRYNRVS

SEQ ID NO: 6 - protein, 7 aa, Artificial Sequence
  CDR2 of light chain
DVTTRPS

SEQ ID NO: 7 - protein, 9 aa, Artificial Sequence
  CDR3 of light chain
GSYAGSYTY

SEQ ID NO: 8 - protein, 5 aa, Artificial Sequence
  CDR1 of heavy chain
DYAIH

SEQ ID NO: 9 - protein, 17 aa, Artificial Sequence
  CDR2 of heavy chain
WINPGSGNTKYSHNFEG

SEQ ID NO: 10 - protein, 12 aa, Artificial Sequence
  CDR3 of heavy chain
SVSAYGSNYFDP

SEQ ID NO: 11 - protein, 11 aa, Artificial Sequence
  CDR1 of light chain
QASQDISNYLN

SEQ ID NO: 12 - protein, 7 aa, Artificial Sequence
  CDR2 of light chain
STSNLQS

SEQ ID NO: 13 - protein, 8 aa, Artificial Sequence
  CDR3 of light chain
QQLNSYPL

SEQ ID NO: 14 - protein, 5 aa, Artificial Sequence
  CDR1 of heavy chain
EHAMH

SEQ ID NO: 15 - protein, 17 aa, Artificial Sequence
  CDR2 of heavy chain
GINWNSGKTGYADSVKG

SEQ ID NO: 16 - protein, 14 aa, Artificial Sequence
  CDR3 of heavy chain
SGGYGGNTNWYFDL

SEQ ID NO: 17 - protein, 14 aa, Artificial Sequence
  CDR1 of light chain
IGTSSNIGVGYDVH

SEQ ID NO: 18 - protein, 7 aa, Artificial Sequence
  CDR2 of light chain
GNNNRPS

SEQ ID NO: 19 - protein, 9 aa, Artificial Sequence
  CDR3 of light chain
QSYDSRLGV

SEQ ID NO: 20 - protein, 5 aa, Artificial Sequence
  CDR1 of heavy chain
DYAMH

SEQ ID NO: 21 - protein, 17 aa, Artificial Sequence
  CDR2 of heavy chian
GISWNSGSIGYADSVKG

SEQ ID NO: 22 - protein, 13 aa, Artificial Sequence
  CDR3 of heavy chain
GPGLATGKGYADY

SEQ ID NO: 23 - protein, 11 aa, Artificial Sequence
  CDR1 of light chain
RASQRISSWLA

SEQ ID NO: 24 - protein, 7 aa, Artificial Sequence
  CDR2 of light chain
SASTLHN

SEQ ID NO: 25 - protein, 8 aa, Artificial Sequence
  CDR3 of light chain
QQGHSFPY

SEQ ID NO: 26 - protein, 5 aa, Artificial Sequence
  CDR1 of heavy chain
LYGMS

SEQ ID NO: 27 - protein, 17 aa, Artificial Sequence
  CDR2 of heavy chain
SIPGSGTRTHYADSVKG

SEQ ID NO: 28 - protein, 8 aa, Artificial Sequence
  CDR3 of heavy chain
STAYLFDY

SEQ ID NO: 29 - protein, 11 aa, Artificial Sequence
  CDR1 of light chain
RASQSIRHYLA

SEQ ID NO: 30 - protein, 7 aa, Artificial Sequence
  CDR2 of light chain
GASSRAT

SEQ ID NO: 31 - protein, 8 aa, Artificial Sequence
  CDR3 of light chain
QHYGSPLH

SEQ ID NO: 32 - protein, 5 aa, Artificial Sequence
  CDR1 of heavy chain
DYYMS

SEQ ID NO: 33 - protein, 17 aa, Artificial Sequence
  CDR2 of heavy chain
YISGSGITTYYADSVKG

SEQ ID NO: 34 - protein, 16 aa, Artificial Sequence
  CDR3 of heavy chain
LQGHCSGGACSNWFDA

SEQ ID NO: 35 - protein, 11 aa, Artificial Sequence
  CDR1 of light chain
RASQSILTYLN

SEQ ID NO: 36 - protein, 7 aa, Artificial Sequence
  CDR2 of light chain
AASSLQR

SEQ ID NO: 37 - protein, 8 aa, Artificial Sequence
  CDR3 of light chain
QQGYGTPY

SEQ ID NO: 38 - protein, 123 aa, Artificial Sequence
  heavy chain VR of B09
QMQLVESGGRLVRPGGSLRLSCAASGFPFTSYAMNWVRQTPGKGLEWVSTITATSGKTYYADSVKGRFTISRDNSRNTLFLQMNSLRAEDTAVYYCVRGESCSGGACSDFDYWGQGALVTVSS

SEQ ID NO: 39 - protein, 113 aa, Artificial Sequence
  light chain VR of B09
QLVLTQPASVSGSPGQSVTISCTGTSSDIGRYNRVSWYQHHPGKAPKLILNDVTTRPSGFSNRFSGSKSGNTASLTISGLQAEDEADYSCGSYAGSYTYVFGTGTKVTVLGGG

SEQ ID NO: 40 - protein, 121 aa, Artificial Sequence
  heavy chain VR of A04
QVQLVESGAEVKKPGASVKVSCKASGYTFKDYAIHWVRQAPGQRLEWMGWINPGSGNTKYSHNFEGRVILTRDASANTAYLELPSLRSEDTAVYYCARSVSAYGSNYFDPWGQGTLITVSS

SEQ ID NO: 41 - protein, 110 aa, Artificial Sequence
  light chain VR of A04
DIQMTQSPSSLSASVGDRVTITCQASQDISNYLNWYQQKPGKAPKLLIYSTSNLQSGVPSRFSGSGSGTDFTLTISSLQPEDFATYYCQQLNSYPLTFGGGTKVDIKRGG

SEQ ID NO: 42 - protein, 123 aa, Artificial Sequence
  heavy chain VR of A05
QMQLVESGGGLVQPGRSLRLSCAASGFTFDEHAMHWVRQAPGKGLEWVSGINWNSGKTGYADSVKGRFTISRDNGKNSLYLQMNSLRAEDTAVYYCAKSGGYGGNTNWYFDLWGRGTLVTVSS

SEQ ID NO: 43 - protein, 113 aa, Artificial Sequence
  light chain VR of A05
QFVLTQPPSVSGAPGQNVTISCIGTSSNIGVGYDVHWYQQVPGTAPKLLVYGNNNRPSGVPDRFSGSKSGTSASLAITGLQAEDEADYYCQSYDSRLGVVFGGGTKLTVLGGG

SEQ ID NO: 44 - protein, 122 aa, Artificial Sequence
  heavy chain VR of A10
QVQLVESGGGLVQPGRSLRLSCAASGFTFDDYAMHWVRQAPGKGLEWVSGISWNSGSIGYADSVKGRFTISRDNAKNSLYLQMNSLRAEDTAVYYCARGPGLATGKGYADYWGQGTLVTVSS

SEQ ID NO: 45 - protein, 110 aa, Artificial Sequence
  light chain VR of A10
DIQMTQSPSSVSASVGDRVTITCRASQRISSWLAWYQQKPGRAPKLLIHSASTLHNGVPSRFSGSASGTDFTLTISSLQPEDYAIYYCQQGHSFPYTFGQGTKLEIKRGG

SEQ ID NO: 46 - protein, 117 aa, Artificial Sequence
  heavy chain VR of H06
QVQLVQSGGGLIQPGGSLRLSCAASGFTFSLYGMSWVRQAPGKGLEWVSSIPGSGTRTHYADSVKGRFTISRDNSKNTLFLQMNSLRAEDTAVYYCAKSTAYLFDYWGPGTLVTVSS

SEQ ID NO: 47 - protein, 110 aa, Artificial Sequence
  light chain VR of H06
DIQMTQSPATLSLSPGERATLSCRASQSIRHYLAWYQQKPGQAPRLLIHGASSRATGIPDRFSGSGSGTDFTLTISRLEPEDFAVYYCQHYGSPLHSFGPGTKVEIKRGG

SEQ ID NO: 48 - protein, 125 aa, Artificial Sequence
  heavy chain VR of H12
QVQLVQSGGGLVKPGGSLRLSCAASEFTFSDYYMSWVRQAPGKGLEWLSYISGSGITTYYADSVKGRFTISRDNGKKSLYLEMNSLRAEDTAVYYCARLQGHCSGGACSNWFDAWGQGTLITVSS

SEQ ID NO: 49 - protein, 110 aa, Artificial Sequence
  light chain VR of H12
DIQMTQSPSSLSASVGDRLTITCRASQSILTYLNWYQQKPGKAPKLLIYAASSLQRGVPSRFSGSGSGTDFTLTISGLQPEDFATYYCQQGYGTPYTFGQGTKVDIKRGG

SEQ ID NO: 50 - DNA, 1416 bp, Artificial Sequence
  polynucleotide sequence coding hevey chain of B09 antibody
atgggatggagctatatcatcctctttttggtggccacagcggccgatgtccactcgcagatgcagctggtggagtctgggggacgcttagtgcggcctggggggtccctgagactctcctgtgcagcctctggattcccctttaccagttatgccatgaactgggtccgccagactccagggaaggggctggaatgggtctctactatcactgctactagtggtaagacatactacgcagactccgtgaagggccggttcaccatctccagagacaactccaggaacacgctgtttctgcaaatgaacagtctgagagccgaggacacggccgtgtattactgtgtgagaggggagtcttgtagtggtggtgcctgctcagattttgactactggggccagggagccctggtcaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctctttcccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 51 - DNA, 717 bp, Artificial Sequence
  polynucleotide sequence coding light chain of B09 antibody
atgggatggagctatatcatcctctttttggtggccacagcggccgatgtccactcgcagctcgtgctgactcagcctgcctccgtgtctgggtctcctggacagtcggtcaccatctcctgcactggaaccagcagtgacattggtcgttataaccgtgtctcctggtaccaacaccaccccggcaaggcccccaaactcattcttaatgatgtcactactcggccctcagggttttctaatcgcttctctggctccaagtctggcaacacggcctccctgaccatctctgggctccaggctgaggatgaggctgattattcctgcggctcatatgcaggcagctacacttatgtcttcggaactgggaccaaggtcaccgtcctaggtggaggaagatctgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 52 - DNA, 23 bp, Artificial Sequence
  forward primer
ctagataacgagggcaaatcatg

SEQ ID NO: 53 - DNA, 19 bp, Artificial Sequence
  reverse primer
cgtcaccaatgaaaccatc

SEQ ID NO: 54 - DNA, 18 bp, Artificial Sequence
  CMV-proF primer
aaatgggcggtaggcgtg

SEQ ID NO: 55 - DNA, 50 bp, Artificial Sequence
  NATVH3-2 primer
ttggtggccacagcggccgatgtccactcgcaggtgcagctggtggagtc

SEQ ID NO: 56 - DNA, 50 bp, Artificial Sequence
  NATVH7-1 primer
ttggtggccacagcggccgatgtccactcgcagatgcagctggtggagtc

SEQ ID NO: 57 - DNA, 50 bp, Artificial Sequence
  NATVH1-1 primer
ttggtggccacagcggccgatgtccactcgcaggtgcagctggtgcagtc

SEQ ID NO: 58 - DNA, 26 bp, Artificial Sequence
  NATJH-ALL primer
gaggaggctagctgaggagacggtga

SEQ ID NO: 59 - DNA, 53 bp, Artificial Sequence
  NATVK6 primer
ttggtggccacagcggccgatgtccactcggacatccagatgacccagtctcc

SEQ ID NO: 60 - DNA, 50 bp, Artificial Sequence
  NATVL13 primer
ttggtggccacagcggccgatgtccactcgcagttcgtgctgactcagcc

SEQ ID NO: 61 - DNA, 50 bp, Artificial Sequence
  NATVL10 primer
ttggtggccacagcggccgatgtccactcgcagctcgtgctgactcagcc

SEQ ID NO: 62 - DNA, 30 bp, Artificial Sequence
  NATJK-R7 primer
gaggagagatcttttgatatccaccttggt

SEQ ID NO: 63 - DNA, 33 bp, Artificial Sequence
  NATJL2-R primer
gaggagagatcttaggacggtcagcttggtccc

SEQ ID NO: 64 - DNA, 30 bp, Artificial Sequence
  NATJK-R5 primer
gaggagagatcttttgatttccagcttggt

SEQ ID NO: 65 - DNA, 33 bp, Artificial Sequence
  NATJL1-R primer
gaggagagatcttaggacggtgaccttggtccc

SEQ ID NO: 66 - DNA, 30 bp, Artificial Sequence
  NATJK-R4 primer
gaggagagatcttttgatttccaccttggt

(19) United States
(12) Patent Application Publication
(10) Pub. No.: US 2014/0072558 A1
(43) Pub. Date: Mar. 13, 2014

(54) Activin Receptor Type II B Inhibitors Comprising DLK1 Extracellular Water-Soluble Domain

(71) Applicants: Young Woo Park, Yuseung Daejeon (KR); Ki Won Jo, Yuseong-gu Daejeon (KR); Donghee Lee, Yuseung-gu Daejeon (KR); Eun Kyung Lee, Yuseong-gu Daejeon (KR); Seil Jang, Yuseong-gu Daejeon (KR); Chan Woong Park, Yuseong-gu Daejeon (KR); Dong-Jin Kim, Yuseong-gu Daejeon (KR); Hye Nan Kim, Yuseong-gu Daejeon (KR); Yun Jung Park, Yuseong-gu Daejeon (KR); Jae Eun Park, Yuseong-gu Daejeon (KR); Ji Hyun Park, Yuseong-gu Daejeon (KR); Seok Ho Yoo, Seo-gu Daejeon (KR); Myeoung Hee Jang, Daedeok-gu Daejeon (KR)

(72) Inventors: Young Woo Park, Yuseung Daejeon (KR); Ki Won Jo, Yuseong-gu Daejeon (KR); Donghee Lee, Yuseung-gu Daejeon (KR); Eun Kyung Lee, Yuseong-gu Daejeon (KR); Seil Jang, Yuseong-gu Daejeon (KR); Chan Woong Park, Yuseong-gu Daejeon (KR); Dong-Jin Kim, Yuseong-gu Daejeon (KR); Hye Nan Kim, Yuseong-gu Daejeon (KR); Yun Jung Park, Yuseong-gu Daejeon (KR); Jae Eun Park, Yuseong-gu Daejeon (KR); Ji Hyun Park, Yuseong-gu Daejeon (KR); Seok Ho Yoo, Seo-gu Daejeon (KR); Myeoung Hee Jang, Daedeok-gu Daejeon (KR)

(73) Assignees: ANTIBODY AND RECEPTOR THERAPEUTICS CO., LTD., Yuseong-gu, Deajeon (KR); KOREA RESEARCH INSTITUTE OF BIOSCIENCE AND BIOTECHNOLOGY, Daejeon (KR)

(21) Appl. No.: 14/009,037

(22) PCT Filed: Apr. 3, 2012
(86) PCT No.: PCT/KR2012/002492

§ 371 Date: Nov. 21, 2013

(30) Foreign Application Priority Data
Apr. 4, 2011 (KR) 10-2011-0030871
Jan. 5, 2012 (KR) 10-2012-0001452

(51) Int. Cl. A61K 39/395; G01N 33/68; A61K 38/17

(52) U.S. Cl. CPC A61K 39/3955; A61K 38/1709; G01N 33/6893
USPC 424/134.1; 514/19.3; 424/139.1; 514/19.8; 514/19.4; 514/4.8; 514/6.9; 514/16.6; 530/350; 530/387.3; 435/7.23; 435/7.21

(57) ABSTRACT

The present invention relates to an activin receptor type II B (ACVR2B) inhibitor which comprises the delta-like 1 homolog (DLK1) extracellular water-soluble domain. More specifically, the present invention relates to an extracellular soluble domain of DLK1; fragments of the extracellular soluble domain of DLK1; mutants of the extracellular soluble domain of DLK1; a composition for suppressing ligand linkage with the ACVR2B receptor, which includes a fragment of the mutants as an active ingredient; and a pharmaceutical composition for prevention and treatment of diseases which comprises the same. The composition of the present invention competitively binds to the ACVR2B receptor and inhibits the binding of an ACVR2B ligand to the ACVR2B receptor, which inhibits protein signalling associated with such ligands, and will be useful for prevention and treatment of diseases associated therewith.

### TECHNICAL FIELD

The present invention relates to an activin receptor-type II B inhibitor comprising an extracellular water-soluble domain of delta-like 1 homolog (DLK1). Particularly, the present invention relates to a composition for suppressing binding between activin receptor type II B (ACVR2B) and a ligand having ACVR2B as a receptor, which includes an extracellular water-soluble domain of DLK1, a fragment of the extracellular water-soluble domain of DLK1, a mutant of the extracellular water-soluble domain of DLK1 or a fragment of the mutant as an active ingredient, and a pharmaceutical composition for preventing or treating a disease including the same.

### BACKGROUND OF THE INVENTION

Cancer is characterized by “uncontrolled cell growth,” and a mass of cells known as a tumor is formed by abnormal cell growth, penetrates into peripheral tissues, and can migrate to different organs of the body when it becomes worse. Such cancer is an intractable chronic disease which is often difficult to cure through treatment by surgery, radiation, and chemotherapy, causes pain to a patient, and ultimately leads to death.

Over the world, over 20,000,000 patients are suffering from cancer, over 6,000,000 patients die of cancer annually, and approximately 11,000,000 patients are expected to die of cancer in 2020, and therefore cancer is a major disease whose therapeutic method should be rapidly found. While there are differences in various countries, cancer is over 20% of all causes of death in the developed countries or Korea. Cancer is roughly classified into blood cancer and solid cancer, and occurs in almost every part of the body as lung cancer, breast cancer, oral cancer, liver cancer, uterine cancer, esophagus cancer, skin cancer, etc. Among methods used to treat malignant tumors other than surgery or radiotherapy, chemotherapies are generally called anti-cancer drugs, which are mainly materials suppressing nucleic acid from exhibiting anticancer activities.

Meanwhile, DLK1 included in the notch/delta/serrate family is a transmembrane glycoprotein encoded in a dlk1 gene located in chromosome 14q32, and composed of 383 amino acids. The protein is divided into 280 extracellular regions, 24 transmembrane regions, and 56 intracellular regions. Among these regions, in the outside of the cell membrane, 6 epidermal growth factor-like repeat domains, 3 N-glycosylation sites and 7 O-glycosylation sites are present. DLK1 is well known as a membrane protein, and also as a protein shed from the cell membrane by a tumor necrosis factor alpha converting enzyme (TACE) and having a separate function.

According to research on the relevance of DLK1 to cancer, it was reported that when DLK1 is overexpressed, and cDNA of DLK1 is overexpressed in glioma, proliferation of the glioma is increased, and migration is increased (Yin D et al., Oncogene, 25: 1852-1861, 2006), the expression of DLK1 in liver cancer is increased compared to normal liver cells, and when the expression of DLK1 is decreased by a siRNA test, a size of the tumor is decreased (Huang J et al., Carcinogenesis, 28(5): 1094-1103, 2007).

In addition, the inventors first established in Korean Patent No. 10-0982170 that an extracellular water-soluble domain of DLK1 and a DLK1-Fc fusion protein formed by a junction of the domain and an Fc region of the IgG antibody can exhibit an anti-cancer effect. However, the type of mechanism that effects the anti-cancer action of the extracellular water-soluble domain of DLK1 has not been discovered.

As a result of the attempts to determine an anti-cancer action mechanism of the extracellular water-soluble domain of DLK1, the inventors found that the extracellular water-soluble domain of DLK1 exhibits the anti-cancer effect by competitively binding activin to ACVR2B to inhibit activin signal transduction, and thus completed the present invention.

The inventors also confirmed that a human antibody specifically binding to the extracellular water-soluble domain of DLK1 could inhibit migration of cancer cells, and significantly increase binding strength of the extracellular water-soluble domain of DLK1 to the ACVR2B, and thus completed the present invention.

### DISCLOSURE

### Technical Problem

The present invention is directed to providing a composition for suppressing the binding between ACVR2B and a ligand having ACVR2B as a receptor, which includes an extracellular water-soluble domain of DLK1 as an active ingredient.

The present invention is also directed to providing a pharmaceutical composition for preventing or treating a disease involved with signal transduction of ACVR2B, which includes the above composition.

The present invention is also directed to providing a kit for diagnosing a disease involved with signal transduction of ACVR2B, which includes the above composition.

### Technical Solution

One aspect of the present invention provides a composition for suppressing binding between ACVR2B and a ligand having ACVR2B as a receptor, which includes an extracellular water-soluble domain of DLK1, a fragment of the extracellular water-soluble domain of DLK1, a mutant of the extracellular water-soluble domain of DLK1, or a fragment of the mutant as an active ingredient.

In the present invention, the term “DLK1” refers to a transmembrane glycoprotein composed of 383 amino acids encoded by a dlk1 gene located in chromosome 14q32.

In the present invention, the term “extracellular water-soluble domain of DLK1” refers to an extracellular water-soluble domain of a DLK1 protein divided into extracellular, transmembrane, and intracellular regions, and an anti-cancer effect of the extracellular water-soluble domain of DLK1 is primarily determined by the inventors.

In the specification, the extracellular water-soluble domain of DLK1 may be expressed in combination with water-soluble DLK1.

Preferably, the water-soluble DLK1 of the present invention is composed of 200 to 300 amino acids having a water-soluble DLK1 activity, and more preferably, comprises an amino acid sequence set forth in SEQ. ID. NO: 1. Any one of amino acid sequences exhibiting an activity of water-soluble DLK1 may be included without limitation.

In the present invention, the term “activin” is a kind of peptide hormone having a molecular weight of approximately 25,000, and classified into three kinds: activin A, which is a homo dimer (βAβA) of a βchain of inhibin A; activin B, which is a homo dimer (βBβB) of a β chain of inhibin B; and activin AB, which is a hetero dimer (βAβB) thereof. Preferably, the activin is activin A.

In the present invention, the term “ACVR2B” is a protein encoded by an ACVR2B gene, and involved with activin signal transduction. It is reported that the signal transduction by activin acts to prevent proliferation of cancer cells and as a tumor inhibitor through cell death, but in contrast, also acts as a pro-oncogenic factor.

As ligands having ACVR2B as a receptor of the present invention, activins (activin A, activin B and inhibin A; Derynck, Zhang et al. 1998), GDF-8/myostatin (Lee and McPherron 2001), Nodal (Oh and Li 2002), BMP6 (Ebisawa, Tada et al. 1999), BMP7 (Yamashita, ten Dijke et al. 1995), GDF5 (Nishitoh, Ichijo et al. 1996), and GDF11 (Oh, Yeo et al. 2002) are known. When such a ligand binds to ACVR2B, it also binds to a type I receptor, ACVR1A or ACVR1B, thereby forming a complex. Here, ACVR2B is phosphorylated to activate the type I receptor. The activated type I receptor activates a transcriptional regulatory factor, Smad, thereby performing cell signaling (Derynck, Zhang et al. 1998).

The signaling of the ligands by ACVR2B is performed by various mechanisms, and it is reported that if the signaling is not suitably regulated, particularly, the ligand is overexpressed, and signal transduction is over-activated, leading to a variety of disorders. Among the disorders, representative ones are as follows:

Generally, it is well known that activin prevents proliferation of cancer cells through apoptosis, but when activin signaling is over-activated, the cancer cells become more aggressive, thereby further stimulating migration. It is a well known phenomenon in TGF-beta (Tang et al., 2003). As the representative example, it was reported that activin A was overexpressed in colorectal cancer stage 4 (Wildi et al., Gut 49:409-417, 2001), and it is known that the overexpression of activin A causes uterine endometrial adenocarcinoma (Tanaka et al., Oncol Rep 11:875-879, 2004). In addition, it is known that activin A induces overexpression of N-cadherin in cancer cells, which makes the cancer aggressive, and thus prognosis of the cancer becomes worse (Yoshinaga et al., Clin Cancer Res, 10:5702-5707, 2004). In addition, in cancer induced by ERBB2, activin signal transduction is considered as a leading edge (Landis M D et al., Oncogene, 24: 5173-5190, 2005), and an increase of invasion by activin A in uterine cancer cell line is also reported (Steller M D et al., Mol Cancer Res, 3: 50-61, 2005).

In addition, activin A plays a major role in glycolysis, and plays different roles in main organs such as the liver, muscle cells, and adipocytes. First, it is reported that activin A suppresses the proliferation of liver cells (Yasuda et al., 1993, De Bleser et al 1997), stimulates glycogenolysis, and stimulates gluconeogenesis, thereby increasing a glucose concentration in blood (Mine et al., 1989). It is known that, in muscle cells, overexpression of activin A brings about muscle atrophy in rats (Gilson et al, 2009), and when activin A is treated, formation of myotubes is prevented (Link and Nishi, 1997). In addition, it is well known that activin A suppresses differentiation in the adipocytes.

In addition, it is known that activin A is expressed in Th2 cells (Ogawa et al., 2006), B cells (Ogawa et al., 2008), alveolar macrophages (Matsuse et al., 1995), peritoneal macrophages (Ogawa et al., 2000), monocyte-derived dendritic cells (Robson et al., 2008), peripheral blood myeloid dendritic cells (Robson et al., 2008), and mast cells (Funaba et al., 2003), and activates immune cells. Accordingly, as activin A is suppressed, autoimmune reaction and inflammatory reaction, immunity-related diseases such as rheumatism, and fibrosis generated by occurrence of inflammation can be prevented and treated.

In addition, the overexpression of activin A induces gonadal tumor development and cachexia, and is accompanied with symptoms such as anemia, weight loss, focal necrosis, inflammation of the liver, and atrophy of the stomach (Matzuk et al., 1994).

Moreover, activin A is known to have an effect on rheumatoid arthritis, bone disorders, sepsis, inflammatory bowel disease, atherosclerosis, and chronic heart failure (Chen et al., 2002; Smith et al., 2004; Werner and Alzheimer, 2006; Yndestad et al., 2004).

It is reported that activin A concentration in blood is high in patients of chronic viral hepatitis and alcohol-induced cirrhosis (Patella et al., 2001; Yuen et al., 2002), and patients of acute liver failure (Hughes and Evans, 2003) and liver cancer (Deli et al., 2008).

It is also reported that activin A concentration in blood is also high in patients of non-alcoholic fatty liver disease (NAFLD) and non-alcoholic steatohepatitis (NASH), and occurrence of fatty liver is induced by activin A (Yndestad et al., 2009). In addition, it is reported that activin A induces hepatic fibrosis (Yndestad et al., 2009).

With reference to BMP6 known as another ligand of ACVR2B, it is known that BMP6 expression is increased in the hippocampus of Alzheimer's patients and in an amyloid-β (Aβ) precursor protein-overexpressed mouse (APP transgenic mouse; Leslie Crews et al., 2010), and overexpression of BMP6 delays reepitheliazation of scars and scar formation in a BMP6-overexpressed mouse (Sibylle Kaiser et al., 1998). In addition, it is reported that BMP-6 is overexpressed in prostate cancer and makes the cancer worse, leading to bone metastasis, and thus the prognosis of the cancer is not good (Ye L et al., 2007, S Darby et al., 2008). Accordingly, it is known that suppression of signal transduction of BMP6 also shows a variety of indications.

Thus, since the composition for suppressing binding according to the present invention effectively inhibits binding between activin involved in a variety of cancers and ACVR2B, it may be useful in preventing and treating cancer. Preferably, the composition for suppressing binding according to the present invention may be useful in preventing and treating skin cancer, breast cancer, uterine cancer, colorectal cancer, kidney cancer, liver cancer, lung cancer, ovarian cancer, or gastric cancer, but the present invention is not limited thereto.

In addition, the composition for suppressing binding according to the present invention may be useful in preventing and treating metabolic diseases, immunological disorders, and liver diseases. Preferably, the composition for suppressing binding according to the present invention may be useful preventing and treating diabetes, obesity, autoimmune diseases, rheumatoid arthritis, chronic hepatitis, alcoholic cirrhosis, acute liver failure, liver cancer, or fatty liver, but the present invention is not limited thereto.

An effect of the composition for suppressing binding according to the present invention for suppressing binding between ACVR2B and the ligand may be achieved by competitively binding water-soluble DLK1 to the ACVR2B in combination with the ligands of the ACVR2B.

Preferably, the composition for suppressing binding according to the present invention includes water-soluble DLK1 comprising a sequence of 200 to 300 amino acids, and more preferably, includes water-soluble DLK1 comprising the amino acid sequence set forth in SEQ. ID. NO: 1.

In addition, the composition for suppressing binding according to the present invention may include a water-soluble DLK1-Fc fusion protein in contact with an antibody Fc region.

In addition, the composition for suppressing binding according to the present invention may further include a human antibody specifically binding to a DLK1 extracellular water-soluble domain or a fragment including an antigen binding site thereof. The human antibody or a fragment including an antigen binding site thereof in the present invention may significantly increase a binding strength of the water-soluble DLK1 to ACVR2B.

In addition, the composition for suppressing binding according to the present invention may further include a known antibody with respect to the ACVR2B, for example, an R&D AF339 antibody.

Another aspect of the present invention provides a human antibody specifically binding to an extracellular water-soluble domain of DLK1 or a fragment including an antigen binding site thereof.

In the present invention, the term “human antibody” refers to a molecule derived from a human immunoglobulin, and all amino acid sequences comprising an antibody including a complementary determining region, and a structural region.

Generally, one antibody molecule has two heavy chains and two light chains, and each of the heavy chains and light chains includes a variable domain at the N-terminal end thereof. Each variable domain is composed of three complementary determining regions (CDRs) and four framework regions (FRs), in which the CDRs determine antigen-binding specificity of the antibody and are present as a relatively short peptide sequence maintained by FRs of the variable domain.

The human antibody or a fragment including an antigen binding site thereof according to the present invention has an activity of specifically binding to water-soluble DLK1. In addition, the human antibody or a fragment including an antigen binding site thereof according to the present invention specifically binds to the water-soluble DLK1, and ultimately has an activity of increasing a binding strength of the water-soluble DLK1 with respect to the ACVR2B. Accordingly, the human antibody or a fragment including an antigen binding site thereof according to the present invention increases a binding strength of an extracellular water-soluble domain of DLK1 with respect to the ACVR2B to prevent binding another ligand capable of competitively binding to DLK1 to the ACVR2B, and thus signal transduction can be suppressed.

In addition, the human antibody or a fragment including an antigen binding site thereof according to the present invention can suppress intracellular signal transduction in a hypoxic state.

In one exemplary embodiment of the present invention, representatively, as it was confirmed that a B09 antibody among the human antibodies of the present invention can bind to water-soluble DLK1, the human antibody of the present invention can specifically bind to the water-soluble DLK1 (referred to FIGS. 21, 22 and 27), and it was confirmed that when the human antibody of the present invention is treated in combination with the water-soluble DLK1, a binding strength of the water-soluble DLK1 with respect to the ACVR2B is significantly increased (referred to FIGS. 25, 27 and 29).

In addition, in one exemplary embodiment of the present invention, as it was confirmed that the B09 antibody among the human antibodies of the present invention can suppress intracellular signal transduction in a hypoxic state, the human antibody of the present invention can suppress the reaction of cells in a hypoxia state (referred to FIG. 28).

Preferably, the human antibody or a fragment including an antigen binding site thereof according to the present invention may be a human antibody or a fragment including an antigen binding site thereof including any one selected from the group consisting of the heavy chain CDR1 sequences set forth in SEQ. ID. NO: 2(SYAMN), SEQ. ID. NO: 8(DYAIH), SEQ. ID. NO: 14(EHAMH), SEQ. ID. NO: 20(DYAMH), SEQ. ID. NO: 26(LYGMS) and SEQ. ID. NO: 32(DYYMS); any one selected from the group consisting of the heavy chain CDR2 sequences set forth in SEQ. ID. NO: 3(TITATSGKTYYADSVKG), SEQ. ID. NO: 9(WINPGSGNTKYSHNFEG), SEQ. ID. NO: 15(GINWNSGKTGYADSVKG), SEQ. ID. NO: 21(GISWNSGSIGYADSVKG), SEQ. ID. NO: 27(SIPGSGTRTHYADSVKG) and SEQ. ID. NO: 33(YISGSGITTYYADSVKG); or any one selected from the group consisting of the heavy chain CDR3 sequences set forth in SEQ. ID. NO: 4(GESCSGGACSDFDY), SEQ. ID. NO: 10(SVSAYGSNYFDP), SEQ. ID. NO: 16(SGGYGGNTNWYFDL), SEQ. ID. NO: 22(GPGLATGKGYADY), SEQ. ID. NO: 28(STAYLFDY) and SEQ. ID. NO: 34(LQGHCSGGACSNWFDA) as a part of a heavy chain variable domain, or a human antibody or a fragment including an antigen binding site thereof according to the present invention including any one selected from the group consisting of the light chain CDR1 sequences set forth in SEQ. ID. NO: 5(TGTSSDIGRYNRVS), SEQ. ID. NO: 11(QASQDISNYLN), SEQ. ID. NO: 17(IGTSSNIGVGYDVH), SEQ. ID. NO: 23(RASQRISSWLA), SEQ. ID. NO: 29(RASQSIRHYLA) and SEQ. ID. NO: 35(RASQSILTYLN); any one selected from the group consisting of light chain CDR2 sequences set forth in SEQ. ID. NO: 6(DVTTRPS), SEQ. ID. NO: 12(STSNLQS), SEQ. ID. NO: 18(GNNNRPS), SEQ. ID. NO: 24(SASTLHN), SEQ. ID. NO: 30(GASSRAT) and SEQ. ID. NO: 36(AASSLQR); or any one selected from the group consisting of the light chain CDR3 sequences set forth in SEQ. ID. NO: 7(GSYAGSYTY), SEQ. ID. NO: 13(QQLNSYPL), SEQ. ID. NO: 19(QSYDSRLGV), SEQ. ID. NO: 25(QQGHSFPY), SEQ. ID. NO: 31(QHYGSPLH) and SEQ. ID. NO: 37(QQGYGTPY) as a part of a light chain variable domain.

More preferably, the human antibody or a fragment including an antigen binding site thereof according to the present invention may include a heavy chain variable domain including a heavy chain CDR1 set forth in SEQ. ID. NO: 2, a heavy chain CDR2 set forth in SEQ. ID. NO: 3 and a heavy chain CDR3 set forth in SEQ. ID. NO: 4; a heavy chain variable domain including a heavy chain CDR1 set forth in SEQ. ID. NO: 8, a heavy chain CDR2 set forth in SEQ. ID. NO: 9 and a heavy chain CDR3 set forth in SEQ. ID. NO: 10; a heavy chain variable domain including a heavy chain CDR1 set forth in SEQ. ID. NO: 14, a heavy chain CDR2 set forth in SEQ. ID. NO: 15 and a heavy chain CDR3 set forth in SEQ. ID. NO: 16; a heavy chain variable domain including a heavy chain CDR1 set forth in SEQ. ID. NO: 20, a heavy chain CDR2 set forth in SEQ. ID. NO: 21 and a heavy chain CDR3 set forth in SEQ. ID. NO: 22; a heavy chain variable domain including a heavy chain CDR1 set forth in SEQ. ID. NO: 26, a heavy chain CDR2 set forth in SEQ. ID. NO: 27 and a heavy chain CDR3 set forth in SEQ. ID. NO: 28; or a heavy chain variable domain including a heavy chain CDR1 set forth in SEQ. ID. NO: 32, a heavy chain CDR2 set forth in SEQ. ID. NO: 33 and a heavy chain CDR3 set forth in SEQ. ID. NO: 34 as the heavy chain variable domain.

In addition, as the light chain variable domain, the human antibody or a fragment including an antigen binding site thereof according to the present invention may include a light chain variable domain including a light chain CDR1 set forth in SEQ. ID. NO: 5, a light chain CDR2 set forth in SEQ. ID. NO: 6 and a light chain CDR3 set forth in SEQ. ID. NO: 7; a light chain variable domain including a light chain CDR1 set forth in SEQ. ID. NO: 11, a light chain CDR2 set forth in SEQ. ID. NO: 12 and a light chain CDR3 set forth in SEQ. ID. NO: 13; a light chain variable domain including a light chain CDR1 set forth in SEQ. ID. NO: 17, a light chain CDR2 set forth in SEQ. ID. NO: 18 and a light chain CDR3 set forth in SEQ. ID. NO: 19; a light chain variable domain including a light chain CDR1 set forth in SEQ. ID. NO: 23, a light chain CDR2 set forth in SEQ. ID. NO: 24 and a light chain CDR3 set forth in SEQ. ID. NO: 25; a light chain variable domain including a light chain CDR1 set forth in SEQ. ID. NO: 29, a light chain CDR2 set forth in SEQ. ID. NO: 30 and a light chain CDR3 set forth in SEQ. ID. NO: 31; or a light chain variable domain including a light chain CDR1 set forth in SEQ. ID. NO: 35, a light chain CDR2 set forth in SEQ. ID. NO: 36 and a light chain CDR3 set forth in SEQ. ID. NO: 37.

More preferably, the human antibody or a fragment including an antigen binding site thereof according to the present invention may be a human antibody or a fragment including an antigen binding site thereof including an amino acid sequence set forth in SEQ. ID. NO: 38, SEQ. ID. NO: 40, SEQ. ID. NO: 42, SEQ. ID. NO: 44, SEQ. ID. NO: 46 or SEQ. ID. NO: 48 as the heavy chain variable domain sequence, and a human antibody or a fragment including an antigen binding site thereof including an amino acid sequence set forth in SEQ. ID. NO: 39, SEQ. ID. NO: 41, SEQ. ID. NO: 43, SEQ. ID. NO: 45, SEQ. ID. NO: 47 or SEQ. ID. NO: 49 as a light chain variable domain sequence. In addition, the heavy chain and the light chain may be used alone or in combination depending on a purpose, and various combinations of a plurality of CDR sequences with the light chain and the heavy chain are possibly formed according to a conventional genetic engineering method depending on a purpose of one of ordinary skill in the art.

In the present invention, the term “fragment including an antigen binding site of a human antibody” is a fragment having an immunological activity of a human antibody molecule of the present invention that forms an antigen (water-soluble DLK1)-antibody bond, which ensures an antigen binding function. Examples of the fragment include (i) an Fab fragment composed of a variable domain of a light chain (VL), a variable domain of a heavy chain (VH), a constant domain of a light chain (CL), and a first constant domain of a heavy chain (CH1); (ii) an Fd fragment composed of VH and CH1; (iii) an Fv fragment composed of VL and VH of a monoclonal antibody; (iv) a dAb fragment composed of VH (Ward E S et al., Nature, 341: 544-546, 1989); (v) an isolated CDR region; (vi) an F(ab′)2 fragment, which is a bivalent fragment including two linked Fab fragments; (vii) a single chain Fv (scFv) molecule bound by a peptide linker to form an antigen binding site with VH and VL; (viii) a bispecific single chain Fv dimer (PCT/US92/09965), and (ix) a polyvalent or polyspecific fragment manufactured by gene fusion, that is, a diabody (WO94/13804), but the present invention is not limited thereto.

The human antibody of the present invention may be easily manufactured by a known technique of preparing a monoclonal antibody. For example, a monoclonal antibody may be prepared by forming a hybridoma using a B lymphocyte obtained from an immunized animal, or using a phage display technique, but the present invention is not limited thereto.

Preferably, the human antibody of the present invention is a human antibody produced from a phage display. An antibody library using a phage display technique is a method of expressing an antibody on a phage surface by obtaining an antibody gene from a B lymphocyte without manufacturing a hybridoma. When the phage display technique is used, many conventional difficulties involved with producing a monoclonal antibody by B-cell immortalization may be overcome. A common phage display technique for an antibody may include: 1) inserting a sequence including a variable domain of an antibody of a random sequence into a gene part corresponding to the N-terminal end of a coat protein p of a phage; 2) expressing a fusion protein of a part of a natural coat protein and the variable domain of the antibody encoded by the random sequence; 3) treating an antigen capable of binding to a library of the fusion protein of the antibody; 4) eluting antibody-phage particles binding to an antigen using a molecule having a low pH and binding competitiveness; 5) amplifying an eluted phage in a host cell; 6) repeating the method to obtain a desired volume; and 7) determining a sequence of the variable domain of an antibody having an activity from DNA sequences of phage clones selected by panning.

In this operation, the phage that can be used to manufacture an antibody library may be, for example, fd, M13, f1, If1, Ike, Zj/Z, Ff, Xf, Pf1 or Pf3 phage as a filamentous phage, but the kind of a phage that can be used in the present invention is not limited to the above examples. In addition, as an example of a vector that can be used for hetero gene expression on a surface of the filamentous phage, a phage vector of fUSE5, fAFF1, fd-CAT1 or fdtetDOG or a plasmid vector of pHEN1, pComb3, pComb8, pKRIBB-Fab or pSEX is used, but the present invention is not limited thereto.

In addition, a helper phage that can be used to provide a wild-type coat protein required for successful reinfection of a recombinant phage for amplification may be, for example, M13K07 or VSCM13, but the present invention is not limited thereto.

Still another aspect of the present invention provides a polynucleotide encoding a fragment including a human antibody according to the present invention or an antigen binding site thereof (hereinafter referred to as a “fragment thereof”), and a recombinant vector including the polynucleotide.

The polynucleotide of the present invention is a polynucleotide in which nucleotide monomers are linked in a long chain shape by a covalent bond, that is, a DNA (deoxyribonucleic acid) or RNA (ribonucleic acid) strand having a certain length or more, encoding the human antibody according to the present invention or a fragment thereof. Preferably, the polynucleotide of the present invention is a heavy chain variable domain set forth in SEQ. ID. NO: 50 and a light chain variable domain set forth in SEQ. ID. NO: 51.

In the polynucleotide encoding the human antibody of the present invention or a fragment thereof, due to degeneracy of a codon or in consideration of a codon preferred by an organism to express the human antibody or a fragment thereof, a coding region may be changed in various ways without changing an amino acid sequence of a human antibody or a fragment thereof expressed from the coding region, and a part excluding the coding region may also be changed or modified in various ways without an effect on gene expression. It is well understood by one of ordinary skill in the art that such a changed gene is also included in the scope of the present invention. That is, in the polynucleotide of the present invention, at least one nucleic acid base may be mutated by substitution, deletion, insertion or a combination thereof as long as a resulting product encodes a protein having an identical activity to the original polynucleotide, and the resulting product is also included in the scope of the present invention.

A recombinant vector of the present invention is a means of inserting DNA into a host cell to express the human antibody of the present invention or a fragment thereof in a microorganism. During the manufacture of the recombinant vector, depending on a host cell for a human antibody or a fragment thereof to be produced, an expression regulatory sequence such as a promoter, a terminator or an enhancer, or a sequence for membrane targeting or secretion may be suitably selected, and combined in various ways according to a purpose.

The recombinant vector of the present invention includes, but is not limited to, a plasmid vector, a cosmid vector, a bacteriophage vector and a virus vector. A suitable recombinant vector may include a promoter, an operator, an initiation codon, a termination codon, a polyadenylation signal, an expression regulatory element such as an enhancer, or a signal sequence or leader sequence for membrane targeting or secretion, and may be manufactured in various forms according to a purpose. A promoter of the recombinant vector may be constitutive or inducible. As the signal sequence, when a host is a yeast, an MFα signal sequence, or an SUC2 signal sequence may be used, or when a host is an animal cell, an insulin signal sequence, an α-interferon signal sequence, or an antibody molecule signal sequence may be used, but the present invention is not limited thereto. In addition, the recombinant vector may include a selective marker for selecting a host cell containing a vector, and a replicable recombinant vector may include a replication origin.

Yet another aspect of the present invention provides a transformant transformed with the recombinant vector of the present invention, and a method of preparing a human antibody or a fragment thereof, which includes: (a) preparing a recombinant vector including a polynucleotide encoding the human antibody of the present invention or a fragment thereof; (b) introducing the recombinant vector into a host cell to be transformed into a transformant; and (c) incubating the transformant.

A large amount of the human antibody of the present invention or a fragment thereof may be produced by transforming the recombinant vector of the present invention into a suitable host cell, for example, a yeast cell or an animal cell, and incubating the transformed host cell.

In the present invention, the term “transformation” means that a gene is introduced into a host cell to be expressed in the host cell. The transformed gene may be any one that is inserted into or located outside of a chromosome of the host cell without limitation as long as it is expressed in the host cell. In addition, the gene is a polynucleotide that can encode a polypeptide, including DNA and RNA. The gene may be introduced in any type as long as it can be inserted into and expressed in the host cell. For example, the gene may be introduced into the host cell in a type of an expression cassette, which is a polynucleotide structure including all factors required for self expression. The expression cassette includes a promoter, a transcription termination signal, a ribosome binding site and a translation termination signal, which are usually operably linked to the gene.

The transformation of a recombinant vector may be performed by introducing a recombinant vector containing DNA of the present invention into a host cell by a method known in the art such as transient transfection, microinjection, transduction, cell fusion, calcium phosphate precipitation, liposome-mediated transfection, DEAE dextran-mediated transfection, polybrene-mediated transfection, or electroporation, but the present invention is not limited thereto.

The host cell may be a eukaryotic cell derived from a yeast such as Saccharomyces cerevisiae, an insect cell, a plant cell, or an animal cell, and preferably, the animal cell may be an autologous or allogeneic animal cell. Transformants manufactured by being introduced into autologous or allogeneic animal cells may be used in cell therapy to treat cancer by administration into individuals.

The transformants of the present invention may be incubated by selecting a suitable one of the methods known in the art to produce the human antibody of the present invention or a fragment thereof.

In addition, the composition for suppressing binding according to the present invention may be a pharmaceutical composition for preventing or treating cancer, metabolic diseases, immunological disorders, or liver diseases as described above.

Yet another aspect of the present invention provides a method of treating a disease including administrating the composition for suppressing binding according to the present invention into an individual in which the above disease occurs or may occur.

In the present invention, the term “prevention” refers to all behaviors for suppressing cancer or delaying occurrence of cancer by administration of the pharmaceutical composition of the present invention, and the term “treatment” refers to all behaviors for improving or bettering symptoms caused by cancer by the administration of the pharmaceutical composition of the present invention.

In the present invention, the term “individual” refers to all kinds of animals including humans in which cancer occurs or may occur.

Preferably, the pharmaceutical composition of the present invention may improve or better symptoms of cancer through activation of suppressing migration or invasion of cancer.

The pharmaceutical composition of the present invention may be administered through a common route to reach target tissues or cells. The pharmaceutical composition of the present invention may be directly or indirectly administered by intraperitoneal injection, intravenous injection, intramuscular injection, subcutaneous injection, intradermal injection, oral medication, intrapulmonary administration, rectal administration, or intracellular administration according to a purpose. To this end, the pharmaceutical composition of the present invention may be administered by an optional device for moving an active material to a target cell.

The pharmaceutical composition of the present invention may further include an available carrier. The pharmaceutical composition including a pharmaceutically available carrier may be prepared in a variety of oral or parenteral dosage forms. In formulation, a generally used diluent or excipient such as a filler, an extending agent, a wetting agent, a disintegrating agent or a surfactant is included. Solid formulations for oral medication include tablets, pills, powders, granules, and capsules, and are prepared in combination with at least one excipient, for example, starch, sucrose, lactose and gelatin. Liquid formulations for oral medication may include suspensions, solutions, emulsions, syrups, water and liquid paraffin, which are commonly used as simple diluents, various excipients, for example, wetting agents, extending agents, sweeteners, fragrances, preservatives, etc. Formulations for parenteral administration may include distilled aqueous solutions, non-aqueous solvents, suspensions, emulsions, lyophilized preparations, and suppositories. Non-aqueous solvents or suspensions may include propylene glycol, polyethylene glycol, vegetable oil such as olive oil, injectable estero such as ethyloleate, etc. As a base for suppositories, witepsol, macrogol, tween 61, cacao butter, luarin butter, glycerogelatin can be used.

The pharmaceutical composition of the present invention may have any one of dosage forms selected from the group consisting of pills, tablets, powders, granules, capsules, suspensions, solutions, emulsions, syrups, distilled aqueous solutions, non-aqueous solvents, lyophilized preparations and suppositories.

The pharmaceutical composition of the present invention is administered in a pharmaceutically effective amount. The term “pharmaceutically effective amount” means that a sufficient amount for treating a disease in a reasonable beneficial/dangerous ratio applicable to medical treatment, and a level of the effective amount may be determined according to factors including a kind of individual, severity, age, sex, drug activity, sensitivity to a drug, administration time, an administration route, an exhausting ratio, duration of treatment, and simultaneously-used drugs, and various other factors well known in the medical field.

The pharmaceutical composition of the present invention may be used alone or in combination with surgery, hormone therapy, drug therapy, and methods of using biological reaction regulators to treat cancer.

Yet another aspect of the present invention provides a kit for diagnosing cancer, metabolic diseases, immunological disorders, or liver diseases, which includes an extracellular water-soluble domain of DLK1, a fragment of the extracellular water-soluble domain of DLK1, a mutant of the extracellular water-soluble domain of DLK1, or a fragment of the mutant.

In the present invention, the kit may include an enzyme-linked immunosorbent assay (ELISA) kit or a sandwich ELISA kit, but the present invention is not limited thereto.

### Advantageous Effects

According to the present invention, as an extracellular water-soluble domain of DLK1, a fragment of the extracellular water-soluble domain of DLK1, a mutant of the extracellular water-soluble domain of DLK1, or a fragment of the mutant competitively binds to an ACVR2B with ligands binding to ACVR2B, the binding between a ligand and a receptor is suppressed, and signal transduction of a protein involved with the ligands is suppressed.

### DESCRIPTION OF DRAWINGS

FIG. 1 shows images and graphs showing a migration inhibitory effect of water-soluble DLK1 in pancreatic cancer cell lines (MIA-PaCa-2).

FIG. 2 shows images and graphs showing an inhibitory effect of water-soluble DLK1 on migration in uterine cancer cell lines (HeLa).

FIG. 3 shows images and graphs showing an inhibitory effect of water-soluble DLK1 on migration in pancreatic cancer cell lines (MIA-PaCa-2).

FIG. 4 shows images and graphs showing an inhibitory effect of water-soluble DLK1 on anchorage independence growth in uterine cancer cell line (HeLa).

FIG. 5 shows images and graphs showing an inhibitory effect of water-soluble DLK1 on scratch treatment in pancreatic cancer cell lines (MIA-PaCa-2) and kidney cancer cell lines (786-O).

FIG. 6 shows western blot results to determine an effect of water-soluble DLK1 on signal transduction of actual cells.

FIG. 7 shows western blot and RT-PCR images showing that water-soluble DLK1 is overexpressed in cell lines overexpressing the water-soluble DLK1.

FIG. 8 shows migration and invasion inhibitory effects of water-soluble DLK1 on anchorage independence growth and scratch treatment using cell lines overexpressing the water-soluble DLK1.

FIG. 9 shows results obtained by co-immunoprecipitation showing that a receptor of water-soluble DLK1 is ACVR2B.

FIG. 10 shows results of enzyme immunoassay showing that water-soluble DLK1 specifically binds to ACVR2B.

FIG. 11 shows results of enzyme immunoassay showing that water-soluble DLK1 does not bind to activin A.

FIG. 12 shows results of enzyme immunoassay showing that water-soluble DLK1 competitively binds to ACVR2B with activin.

FIG. 13 shows results of surface plasmon resonance showing that water-soluble DLK1 specifically binds to ACVR2B of various activin receptor families.

FIG. 14 shows western blot images showing expression of cells used in flow cytometry.

FIG. 15 shows results of flow cytometry to detect binding between water-soluble DLK1 and ACVR2B occurring on actual cells.

FIG. 16 shows results of enzyme immunoassay for a polyclonal phage antibody with respect to water-soluble DLK1.

FIG. 17 shows results obtained through finger printing to show diversity of a monoclonal phage clone antibody to water-soluble DLK1.

FIG. 18 shows results obtained by analyzing a polypeptide in a CDR of a monoclonal phage antibody with respect to water-soluble DLK1.

FIG. 19 is a cleavage map of a pNATAB H vector.

FIG. 20 is a cleavage map of a pNATAB L vector.

FIG. 21 shows results of flow cytometry (top) and fluoroimmunoassay results (bottom) to show that the B09 antibody specifically binds to a water-soluble domain of DLK1.

FIG. 22 shows results obtained by measuring a binding strength between water-soluble DLK1 and the B09 antibody by surface plasmon resonance.

FIG. 23 shows results obtained by measuring a binding strength between activin A and ACVR2B by surface plasmon resonance.

FIG. 24 shows results obtained by measuring a binding strength between water-soluble DLK1 and ACVR2B by surface plasmon resonance.

FIG. 25 shows results obtained by measuring a binding strength between water-soluble DLK1 and ACVR2B when the B09 antibody and water-soluble DLK1 are treated together by surface plasmon resonance.

FIG. 26 is a schematic diagram showing an effect of the B09 antibody on a binding strength between water-soluble DLK1 and ACVR2B.

FIG. 27 shows results of enzyme immunoassay showing an effect of an antibody on binding between water-soluble DLK1 and ACVR2B.

FIG. 28 shows results of enzyme immunoassay showing an effect of an antibody on binding between activin A and ACVR2B.

FIG. 29 shows results of enzyme immunoassay showing effects of water-soluble DLK1, the DLK1 antibody and the ACVR2B antibody on binding between activin A and ACVR2B.

FIG. 30 shows western blot images showing an effect of water-soluble DLK1 on activin signal transduction.

FIG. 31 shows results of secretory alkaline phosphatase (SEAP) reporter analysis showing an effect of water-soluble DLK1 on Smad signal transduction.

FIG. 32 shows results of SEAP reporter analysis showing an effect of the B09 antibody on Smad signal transduction.

FIG. 33 shows results of hypoxia response element (HRE) reporter assay showing an inhibitory effect of the DLK1 antibody on signal transduction in a hypoxia state.

FIG. 34 shows inhibitory effects of the ACVR2B antibody and the DLK1 antibody on migration.

FIG. 35 shows effects of the ACVR2B antibody and the DLK1 antibody on a migration inhibitory effect of water-soluble DLK1.

FIG. 36 shows a tumor incidence rate of a DLK1-Fc treated group in a pancreatic cancer orthotopic migration animal model test.

FIG. 37 shows a hernia rate of a DLK1-Fc treated group in a pancreatic cancer orthotopic migration animal model test.

FIG. 38 shows that proliferation of cancer cells is decreased in a DLK1-Fc treated group in a pancreatic cancer orthotopic migration animal model test.

FIG. 39 shows weights and sizes of the pancreas and stomach of a DLK1-Fc treated group in a pancreatic cancer orthotopic migration animal model test.

FIG. 40 shows that an enlarged spleen is decreased in a DLK1-Fc treated group in a pancreatic cancer orthotopic migration animal model test.

FIG. 41 shows in vivo images of a pancreatic cancer orthotopic migration animal model.

### MODE FOR INVENTION

Hereinafter, the present invention will be described in further detail. The following Examples are merely provided to describe the present invention, but the scope of the present invention is not limited thereto.

### Reference Example 1

### Preparation of Water-Soluble DLK1 and DLK1-Fc Fusion Protein

An extracellular water-soluble domain of DLK1(herein after, referred to as a “water-soluble DLK1”) and a DLK1-Fc fusion protein (hereinafter, DLK1-Fc) in which water-soluble DLK1 and a human antibody Fc region are joined, which were used in Example of the present invention, were prepared by the method disclosed in Korean Patent No. 10-0982170.

### Example 1

### Confirmation of Inhibitory Effect of Water-Soluble DLK1 on Cancer Migration

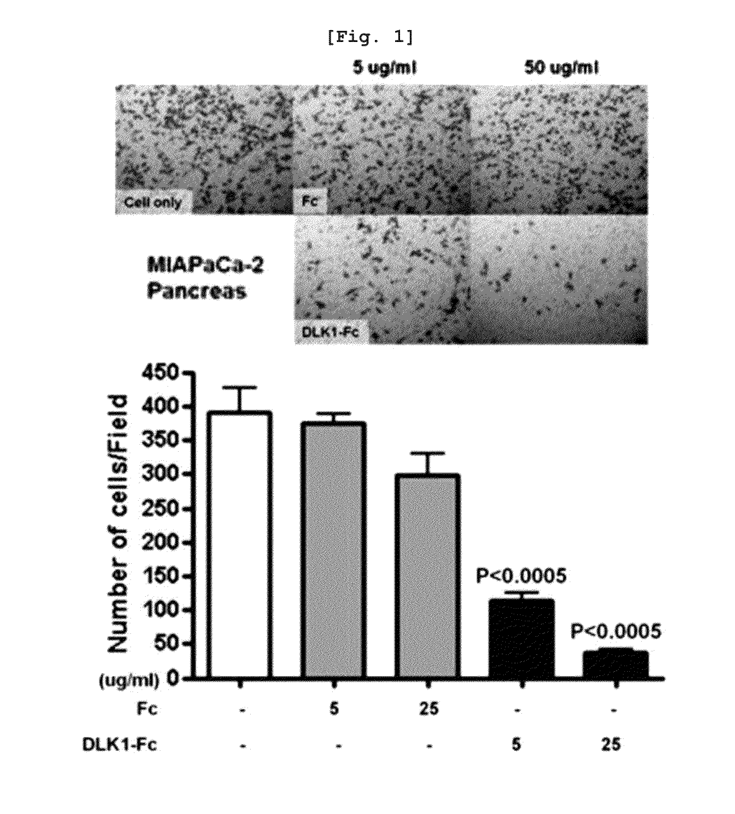

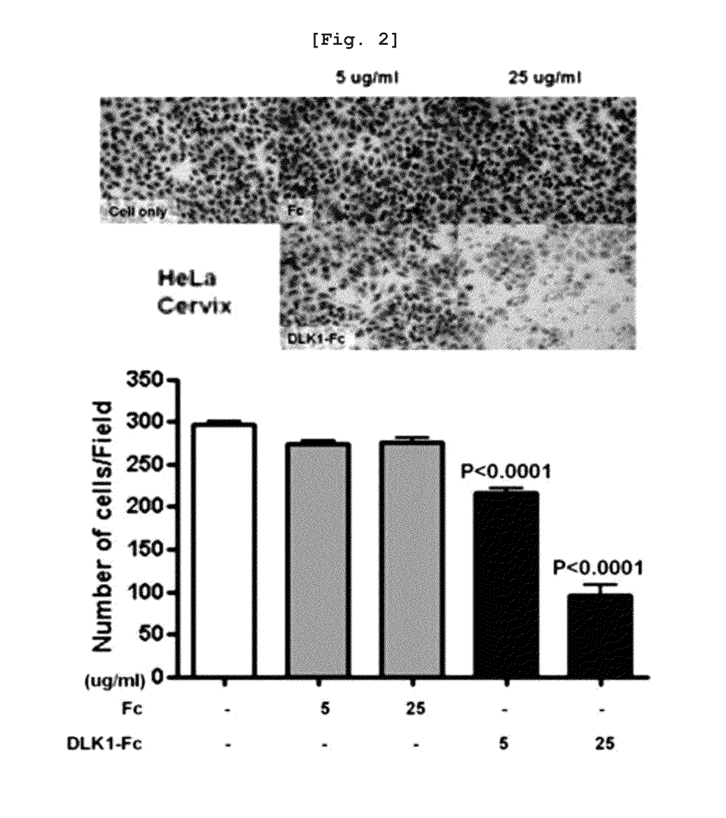

To examine an effect of water-soluble DLK1 on cancer cell lines, a migration assay for cancer cell lines was performed using the method disclosed in Reference (Chen H C, Methods in molecular biology. 294:15-22, 2005). First, when cells (MIA-PaCa-2, HeLa cell line) were grown approximately 50%, a medium was changed with a serum-free medium, cells were detached by treatment with trypsine after 24 hours, and cells were counted. The cells, serum-free medium, and each protein to be treated were mixed to have a total volume of 100 μl, and then incubated at 37° C. for 1 hour. 1 ml of 5˜10% FBS was put into a 24-well plate as a chemo-attractant, a transwell (Corning #3422) having a 8.0 μm pore was put thereon, 100 μl of a solution of mixing pre-cultured cells, cells, and proteins was added for 1 hour, and then the resulting mixture was incubated in a carbon dioxide incubator at 37° C. for 24 to 48 hours. After the incubation, a medium of the transwell was removed, the cells were dyed using a Diff Quick solution (Sysmex, Japan), and the cells not passing through the transwell were completely removed using a swab. Afterward, the transwell was completely dried, and the cells passing through the transwell were enlarged with 400 magnification and photographed, which are shown in FIGS. 1 and 2.

As a result, as shown in FIG. 1, when DLK1-Fc was treated to a pancreatic cancer cell line, MIA-PaCa-2, it was seen that migration of cells was dose-responsively inhibited (FIG. 1). In addition, as shown in FIG. 2, when DLK1-Fc was treated to a uterine cancer cell line, HeLa, it was seen that migration of cells was dose-responsively inhibited (FIG. 2).

### Example 2

### Confirmation of Effect of Water-Soluble DLK1 on Anchorage Independence Growth in Cancer Cell Lines

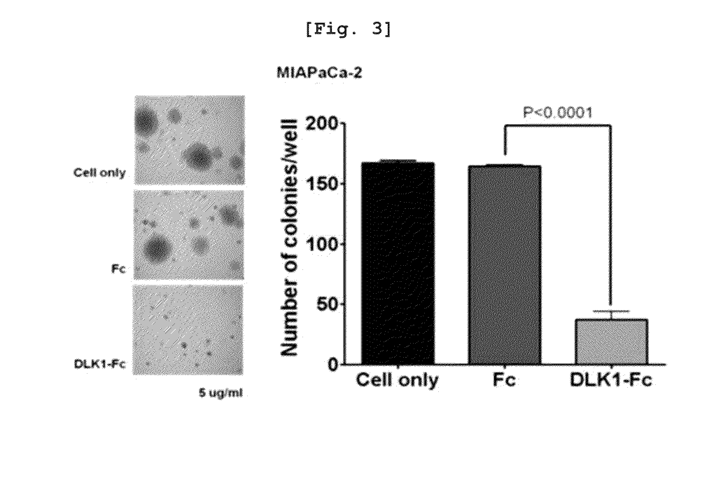

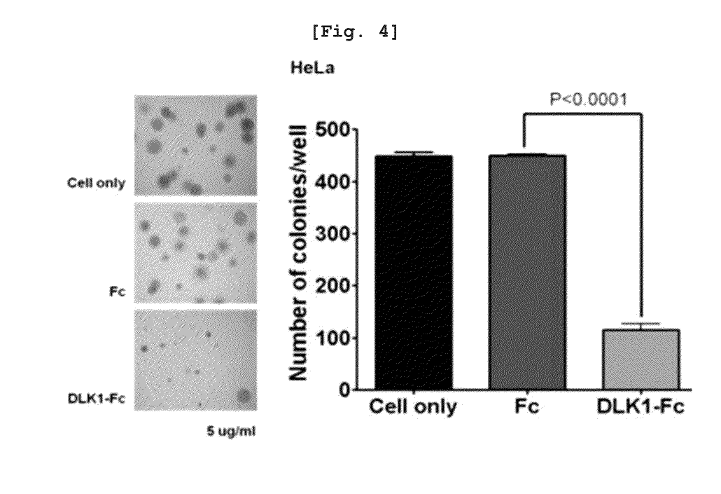

To examine an effect of water-soluble DLK1 on anchorage independence growth, a soft agar assay was performed. First, after an RPMI medium containing 10% FBS and 1% agar were mixed in a ratio of 1:1 and plated on a 60 mm petri dish to have a agar content of 0.5%, 6×10³ of cells (MIA-PaCa-2, HeLa cell line) were mixed with 5 μg/ml of DLK1-Fc or Fc, and an RPMI medium containing 10% FBS and 1% agar were mixed to have a final content of agar of 0.3%, and then plated on the 0.5% agar. The cells were incubated in a CO₂ incubator at 37° C. for 3 weeks, and a medium containing 5 μg/ml of DLK1-Fc or Fc was added every three days to prevent dryness of the agar. After colonies were generated, the colonies were dyed with 0.05% crystal violet and quantified, which are shown in FIGS. 3 and 4.

As a result, when DLK1-Fc was treated to a pancreatic cancer cell line, MIA-PaCa-2, it can be known that anchorage independence growth was inhibited, compared to cells only treated with Fc as a control (FIG. 3). In addition, it can be known that when DLK1-Fc was treated to a uterine cancer cell line, HeLa, compared to the control, cells only treated with Fc, anchorage independence growth was inhibited (FIG. 4).

### Example 3

### Confirmation of Effect of Water-Soluble DLK1 on Scratch Treatment of Cancer Cell Lines

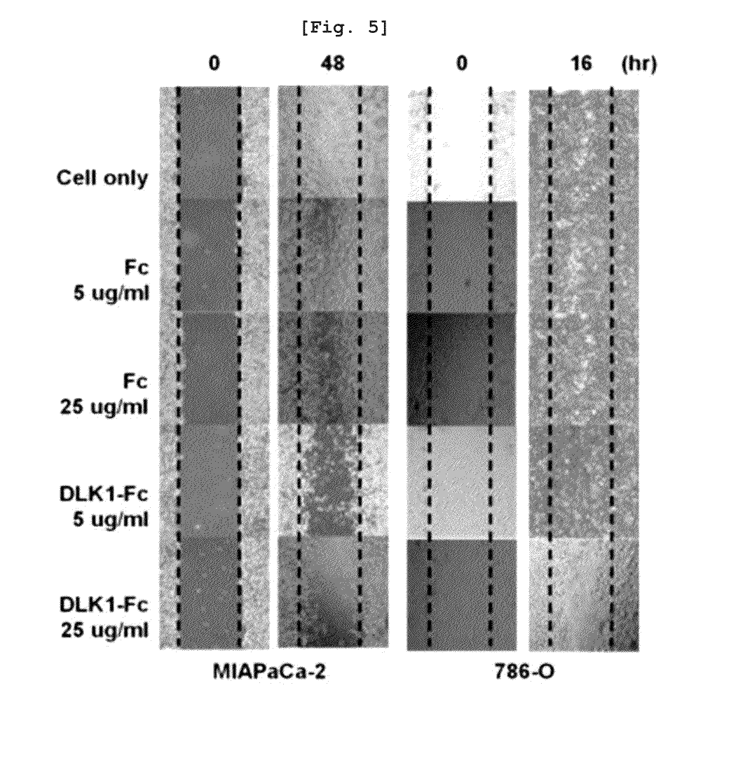

When cells in a pancreatic cancer cell line, that is, MIA-PaCa-2, and a kidney cancer cell line, that is, 786-O, were grown in a 6-well plate approximately 90%, and a medium was changed with a serum-free medium every 16 hours. Afterward, DLK1-Fc or Fc having a concentration of 5˜25 mg/ml was treated to the cells for 1 hour, the cells were scratched, 5% FBS was added, the 786-O cell line was incubated for 16 hours, and the MIA-PaCa-2 cell line was incubated for 48 hours. Coverage of the scratches was observed, which is shown in FIG. 5.

As a result, it can be known that when DLK1-Fc was treated to a pancreatic cancer cell line (MIA-PaCa-2) and a kidney cancer cell line (786-O), compared to the control, which is the cells only treated with Fc, scratch treatment was inhibited (FIG. 5).

### Example 4

### Verification of Signal Transduction by Water-Soluble DLK1

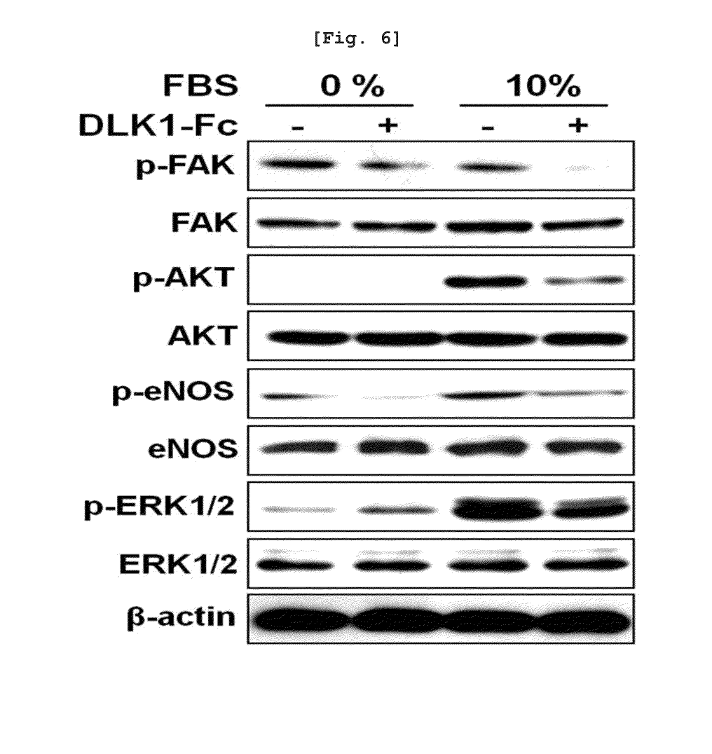

Western blotting was performed to examine an effect of water-soluble DLK1 on signal transduction of an actual cell. To this end, when a pancreatic cancer cell line, MIA-PaCa-2, was grown in a culture container approximately 70%, a medium was changed with a serum-free medium, and the cells were incubated for 16 hours. 10 μg/ml of DLK1-Fc was treated to a DLK1-Fc-treated group, 10% FBS was added, and then the cells were harvested after 10 minutes. The harvested cells were lysed with buffer prepared by mixing a protease inhibitor (Roche), and phosphotase inhibitor cocktails I and II (Sigma) with RIPA buffer (50 mM TrisHCl pH 7.4, 150 mM NaCl, 2 mM EDTA, 1% NP-40), and a concentration of the cell lysate was determined using a BCA quantification kit (Thermo). 30 μg of the lysate was denatured using 5× sample buffer and subjected to electrophoresis using SDS-PAGE, and then transferred to an NC membrane (Watman). The membrane was subjected to western blotting using p-FAK, FAK, p-AKT, AKT, p-eNOS, eNOS, p-ERK1/2, and ERK1/2 antibody (cell signaling). Here, a beta-actin antibody (Sigma) was used as a loading control. The membrane was reacted overnight at 4° C., washed with TBST (Tween 0.05%) three times, reacted with a rabbit-HRP antibody and a mouse-HRP antibody (Santa Cruz) at room temperature for 1 hour, and washed again with TBST (Tween 20 0.05%) three times. Afterward, the blots were labeled with a fluorescence dye in an ECL solution (Intron), and exposed to a film, which is shown in FIG. 6.

As a result, it could be confirmed that p-FAK, p-AKT, and p-eNOS were significantly reduced, and the p-ERK1/2 level was also slightly reduced (FIG. 6). Among these results, the reduction of p-FAK, p-AKT, and p-eNOS supported the effects shown in FIGS. 1 to 5, and the reduction of p-ERK1/2 supported the inhibitory effects on anchorage independence growth shown in FIGS. 3 and 4.

### Example 5

### Construction of Cell Line Stably Expressing Water-Soluble DLK1

To reconfirm the effects of the water-soluble DLK1 treated in an external environment, a cell line overexpressing the water-soluble DLK1 was manufactured using the pancreatic cancer cell line, MIA-PaCa-2. The cell line was constructed by adding a DNA fragment encoding an extracellular domain of DLK1 to a pcDNA 3.1 vector. The cell line was screened in 500 μg/ml G418 (Geneticin, GIBCO/BRL), RT-PCR was performed to confirm whether the cell line was well constructed, and then the expression of the water-soluble DLK1 was confirmed by western blotting after a culture solution of the cell line was concentrated 10 times using Centricon 15 (Millpore). For the western blotting, a DLK1 monoclonal antibody (R&D) was used. As a control, only a vector without an insert was transfected to use.

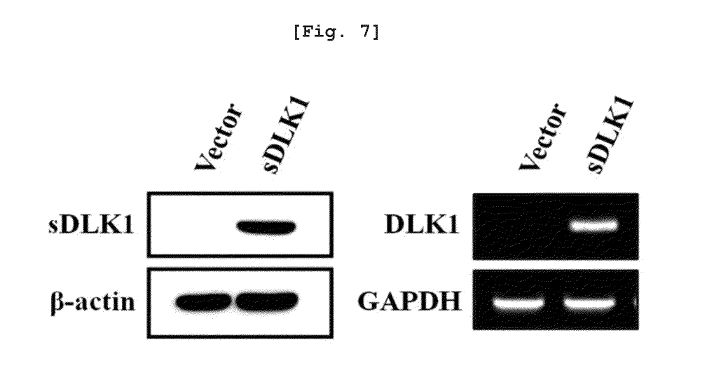

As a result, as shown in FIG. 7, it was confirmed that the water-soluble DLK1 was stably expressed in the cell line (FIG. 7).

### Example 6

### Verification of Inhibitory Effects of Water-Soluble DLK1 on Cancer Migration, Invasion, Anchorage Independence Growth and Scratch Treatment in Cell Lines in which Water-Soluble DLK1 is Stably Expressed

Migration and invasion mechanism were analyzed using the cell line constructed in Example 5. First, when cells were grown approximately 50%, a medium was changed with a serum-free medium, cells were detached by treatment with trypsine after 24 hours, and cells were counted. 1 ml of 5˜10% FBS was put into a 24-well plate as a chemo-attractant, a transwell (Corning #3422) having a 8.0 μm pore was put thereon, 5×10⁴ of cells were put therein, and then the cells were incubated in a carbon dioxide incubator at 37° C. for 24 to 48 hours. After the incubation, the medium of the transwell was removed, the cells were dyed using a Diff Quick solution (Sysmex, Japan), and the cells not passing through the transwell were completely removed using a swab. Afterward, the transwell was completely dried, and the cells passing through the transwell were enlarged with 400 magnification and photographed.

To observe invasion, 100 μl of Matrigel (BD Science) having a concentration of 1 mg/ml was coated on a bottom surface of the transwell, and then an experiment was performed.

To observe anchorage independence growth, an RPMI medium containing 10% FBS and 1% agar were mixed in a ratio of 1:1, and plated on a 60 mm petri dish to have a content of agar of 0.5%, and 6×10³ of cells were added to an RPMI medium containing 10% FBS and 1% agar to have a final content of agar of 0.3%, and plated on the 0.5% agar. The cells were incubated in a carbon dioxide incubator at 37° C. for three weeks, and a medium was transferred every three days to prevent dryness of agar. After formation of colonies, the colonies were photographed, and the results were observed.

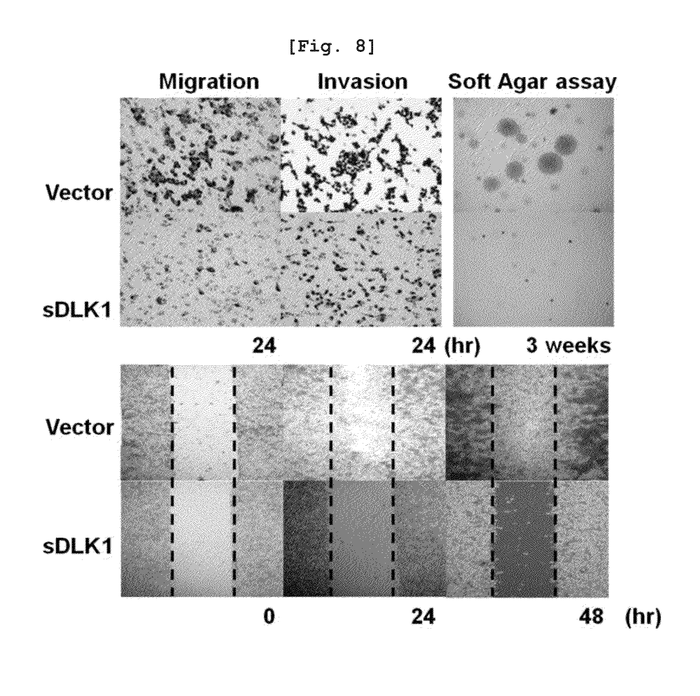

For analysis of scratch treatment, when the cell line was grown approximately 90% on a 6-well plate, the medium was transferred to a serum-free medium. After the cells were incubated in the serum-free medium for 16 hours, the cells were scratched, 5% FBS was added, the cells were further incubated for 48 hours, and then scratch coverage was observed. As a result, as shown in FIG. 8, it was reconfirmed that, like when foreign DLK1-Fc was treated to the cells, migration, invasion, anchorage independence growth, and scratch treatment was suppressed by water-soluble DLK1 expressed in the cell line (FIG. 8).

### Example 7

### Confirmation of Receptor of Water-Soluble DLK1

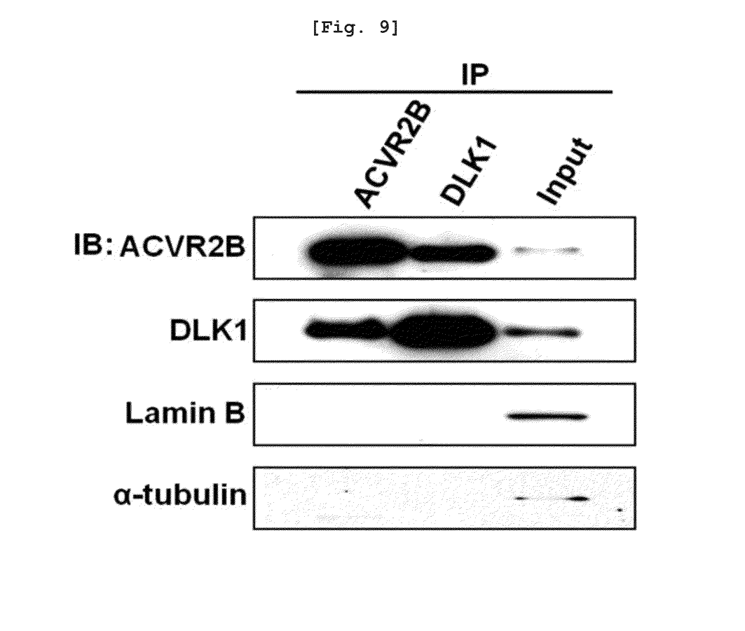

To confirm a receptor of water-soluble DLK1, co-immunoprecipitation was performed. To this end, first, water-soluble DLK1 and ACVR2B were transfected into 293E cells by a PEI (Polyscience) method to overexpress. The cells were washed with cold DPBS three times, and lysed with modified RIPA buffer (50 mM TrisHCl pH7.4, 150 mM NaCl, 2 mM EDTA, 1% NP-40). The lysate was pre-cleared using human IgG (Jackson Lab) and protein G beads (GE). Immunoprecipitation was performed using human antibodies directly manufactured in vitro and the protein G beads, a sample was isolated in SDS-PAGE, and then western blotting was performed. Results are shown in FIG. 9. For western blotting, a DLK1 monoclonal antibody (R&D) and an ACVR2B affinity purified polyclonal antibody (R&D) were used, and as controls, a Lamin B antibody (Santa Cruz) and an α-tubulin antibody (Santa Cruz) were used.

As a result, as shown in FIG. 9, it was confirmed that the water-soluble DLK1 bound to ACVR2B involved with activin signaling (FIG. 9).

### Example 8

### Confirmation of Binding Between Water-Soluble DLK1 and ACVR2B

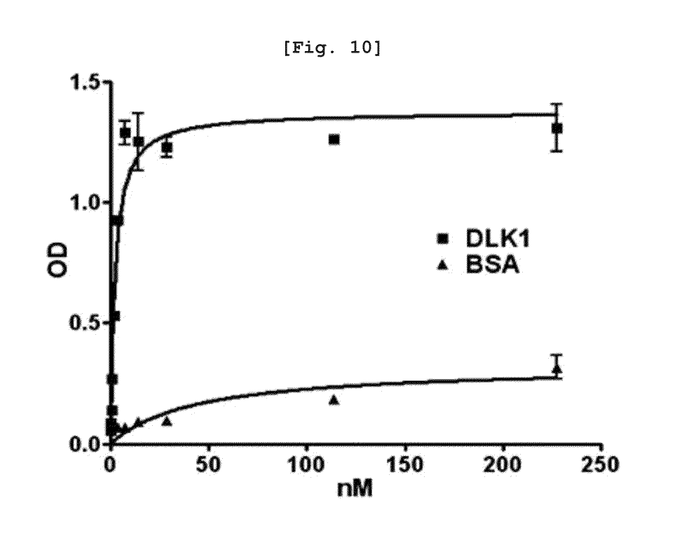

Binding between ACVR2B and water-soluble DLK1 was confirmed by enzyme immunoassay (ELISA). To this end, 1 μg/ml of FLAG-tagged water-soluble DLK1 was coated on an immunoplate (Nunc) at 4° C. for 16 hours. Here, as a control for detecting non-specific binding, BSA was used. The coated plate was washed with PBST (0.05% Tween 20) three times, and blocked with 5% skim milk PBST. ACVR2B-Fc prepared by attaching Fc to an extracellular part of ACVR2B was subjected to serial dilution, and reacted at room temperature for 1 hour. After the reaction, the cells were washed with PBST (0.05% Tween 20) three times, and human IgG-HRP (1:5000) was incubated for 1 hour to measure a binding degree. After the reaction, the cells were washed with PBST (0.05% Tween 20) three times, a color reaction was performed using TMB (Sigma), and 2.5 M H₂SO₄ was added to stop the reaction. And then optical density was measured at 450 nm. As a result, as shown in FIG. 10, it was confirmed that water-soluble DLK1 bound to ACVR2B, but not to BSA (FIG. 10).

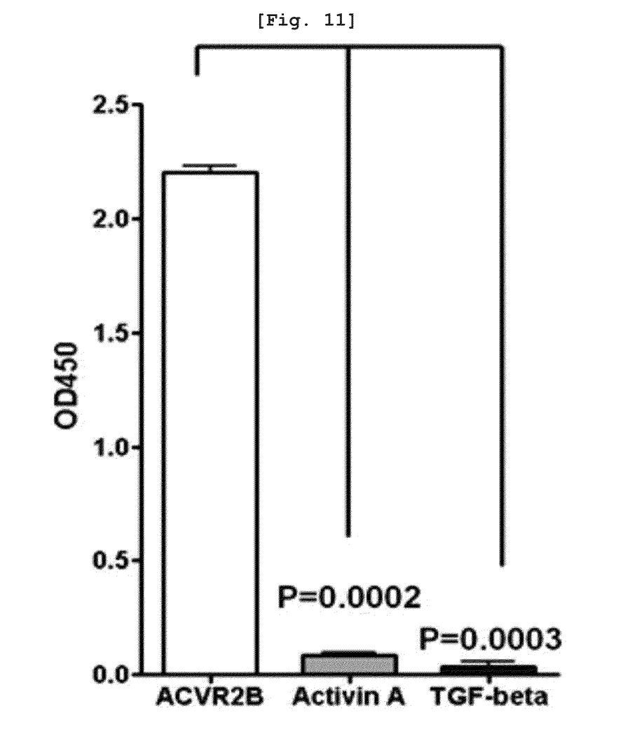

In addition, to examine the relationship between water-soluble DLK1 and a ligand of ACVR2B, activin A, a binding degree with DLK1-Fc was measured after the activin A was coated. As a result, it was confirmed that the water-soluble DLK1 did not bind to the ligand of the ACVR2B, activin A (FIG. 11).

### Example 9

### Confirmation of Competitive Binding Between Water-Soluble DLK1 and ACVR2B with Activin A

To examine whether water-soluble DLK1 competitively bind to the ACVR2B receptor with activin A, enzyme immunoassay was performed. 50 ng/ml of activin A was coated and reacted with ACVR2B-Fc and FLAG-DLK1, and then the binding was detected. Here, ACVR2B was fixed to a concentration of 2 nM, FLAG-DLK1 was treated to have a concentration of 1 to 16 nM, and then a degree of competitive binding was measured. As controls, Fc and ACVR1A-Fc not binding to activin A were used.

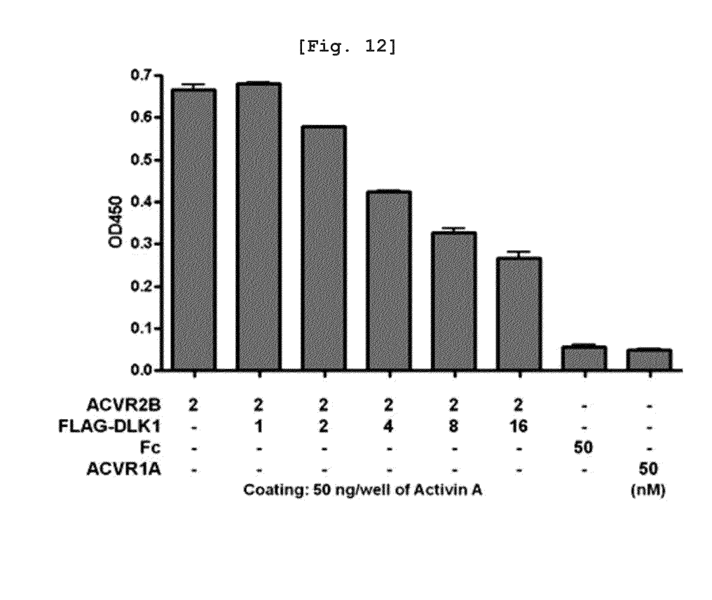

As a result, as shown in FIG. 12, it was seen that as a concentration of water-soluble DLK1 is increased, the binding between activin A and ACVR2B was gradually reduced (FIG. 12). Such results showed that activin signal transduction could be prevented by competitively binding water-soluble DKL1 to ACVR2B with activin A.

### Example 10

### Confirmation of Specific Binding Between Water-Soluble DLK1 and ACVR2B

To confirm the specific binding between water-soluble DLK1 and ACVR2B, surface plasmon resonance was performed using ProteOn XPR36 (Bio-Rad), and the method was summarized as follows. While 0.1 M EDC and 0.025 M sulfo-NHS flew for 60 seconds, a sensor chip was activated, a protein to be coated was added to 10 mM sodium acetate (pH 5.0) to flow for 240 seconds at a rate of 30 μl/min, thereby performing attachment to the chip. A channel was blocked with 1 M ethanolamine-HCl (pH 8.5) for 200 seconds, and washed with DPBST (PBS with 0.005% tween 20). To detect a binding strength, the resulting solution was two times serial diluted, and flew at a rate of 30 μl/min for 120 seconds. To examine a dissociation strength, a dissociation phase was brought for 240 seconds.

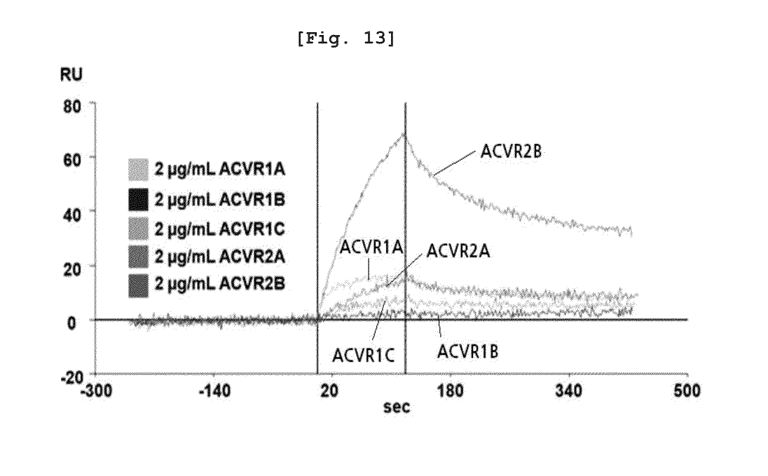

As a result, as shown in FIG. 13, it was confirmed that water-soluble DLK1 specifically bound to ACVR2B among a variety of activin receptor families (FIG. 13).

### Example 11

### Confirmation of Binding Between Water-Soluble DLK1 and ACVR2B on Actual Cells

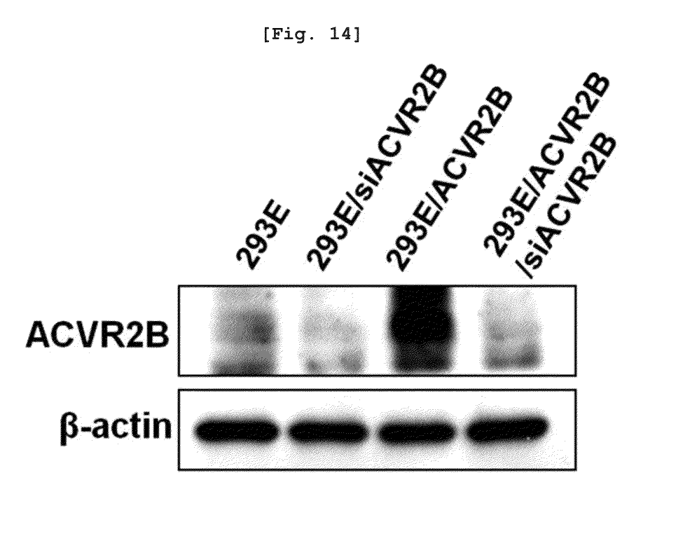

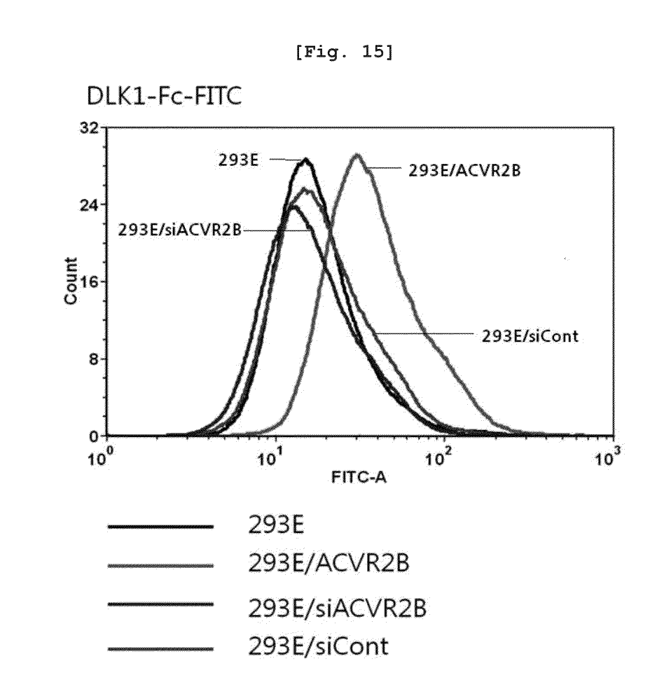

To confirm binding between water-soluble DLK1 and ACVR2B on actual cells, an experiment was performed using flow cytometry. The experiment was performed using CantoII (BD), and an experiment method was summarized as follows. A pcDNA vector including a total gene of ACVR2B was transfected into a 293E cell using a PEI (Polyscience) method, a cell to overexpress ACVR2B was prepared. After two days of the transfection, the transfected result was harvested with a un-transfected 293E cell. In addition, to reconfirm the meaning of the binding between water-soluble DLK1 and ACVR2B, the expression of ACVR2B was suppressed by treating siRNA (Invitrogen) for suppressing the expression of ACVR2B, cells were harvested, and as a control to siRNA, control siRNA (Invitrogen)-treated 293E was also prepared. The harvested cells were washed with cold DPBS twice, 1 μg of DLK1-Fc-FITC was added to react at 4° C. for 2 hours. DLK1-Fc-FITC used in the reaction was manufactured using a protein FITC labeling kit (Pierce). After the reaction, washing with 1% PBA (1% BSA in DPBS) buffer was performed, a degree of binding was determined by flow cytometry, and the results are shown in FIG. 15. In addition, the cells used above were subjected to western blotting to confirm the expression of ACVR2B (FIG. 14).

As a result, as shown in FIG. 15, it was seen that binding migration by water-soluble DLK1 was significantly increased in the ACVR2B-overexpressed cell line, but migration was decreased in the cell line in which the expression of ACVR2B was suppressed by treatment of siRNA (FIG. 15).

### Example 12

### Manufacture and Verification of Human Antibody Specifically Binding to Water-Soluble Domain of DLK1

12-1. Manufacture of Human Antibody Specifically Binding to Water-Soluble Domain of DLK1

<12-1-1> Manufacture of Library Phage

Human-derived scFv library cells having a diversity of 2.7×10¹⁰ were incubated in a medium (3l) including 17 g of 2×YTCM [Tryptone (CONDA, 1612.00), 10 g of Yeast extract (CONDA, 1702.00), 5 g of NaCl (sigma, S7653-5 kg), 34 μg/ml of chloramphenicol (sigma, C0857)], 2% glucose (sigma, G5400), and 5 mM MgCl₂ (sigma, M2393) at 37° C. for 2 to 3 hours (OD600=0.5˜0.7), infected by a helper phage, and incubated in a 2×YTCMK [2×YT CM, 70 μg/ml of Kanamycin (sigma, K1876), 1 mM IPTG (ELPISBIO, IPTG025)] medium at 30° C. for 16 hours. The incubated cells were subjected to centrifugation (4500 rpm, 15 minutes, 4° C.), 4% PEG (Fluka, 81253) 6000 and 3% NaCl (sigma, S7653) were added to and sufficiently melted in a supernatant, and the resulting supernatant was reacted in ice for 1 hour. Centrifugation (8000 rpm, 20 minutes, 4° C.) was performed again, PBS was added to a pellet to melt and centrifuged (12000 rpm, 10 minutes, 4° C.), and a supernatant including a library phage was put into a new tube and stored at 4° C.

<12-1-2> Manufacture of Monoclonal Antibody

(1) Panning Process

30 μg of purified DLK1-Fc yielded in Example 1 was coated with 4 ml of coating buffer [1.59 g of Na₂CO₃ (sigma, S7795), 2.93 g of NaHCO₃ (sigma, S8875), 0.2 g of NaN₃ (sigma, S2002)] on an immunosorb tube (Nunc 470319) using a rotator at 4° C. for 16 hours, melted in PBS at room temperature for 2 hours, and blocked with skim milk [(BD,232100)-4% in 1XPBS] in the immunosorb tube. 2 ml of the manufactured library phage was added to the immunosorb tube to react at room temperature for 2 hours, and washed with PBST (0.05%) five times and with PBS twice. After the washing, only specifically binding scFv-phages were eluted with 100 mM TEA (Sigma T-0886), and the eluted phages were infected to E. coli (XL1-Blue, Stratagene, 200249) to amplify. After the first panning, the amplified phages were subjected to second and third panning by the same method as described above, except that the number of PBST washing was increased (second panning: 13 times, third panning 23 times).

As a result, the increase in potency of antibodies according to panning is shown in Table 1.

TABLE 1

| | Number of | Number of | Number of |
|---|---|---|---|
| Target antigen | panning | initial phages | binding phages |
| DLK1-Fc | 1st | 4.6 × 10¹³ | 6 × 10⁷ |
| | 2nd | 2 × 10¹² | 1.4 × 10⁶ |
| | 3rd | 1.49 × 10¹⁴ | 6.93 × 10⁹ |

(2) Detection of Phage Antibody Using Phage Enzyme Immunoassay (ELISA)

i) Confirmation of Panning Results

A frozen cell stock underwent the first to third panning was added to a medium including 5 ml of 2×YTCM, 2% glucose and 5 mM of MgCl₂ to have OD600 of 0.1, and incubated at 37° C. for 2 to 3 hours (OD600=0.5˜0.7). Afterward, an M1 helper phage was infected thereto, and the resulting stock was incubated in a medium including 2×YTCMK, 5 mM of MgCl₂, and 1 mM IPTG at 30° C. for 16 hours. The incubated cells were centrifuged (4500 rpm, 15 minutes, 4° C.), and a supernatant (panned poly scFv-phage) was transferred to a new tube. After 100 ng/well of antigens were treated with coating buffer at 4 C for 16 hours to coat a 96-well immunoplate (NUNC 439454), each well was blocked with skim milk (4%) dissolved in PBS. Each well was washed with 0.2 ml of PBS-tween20 (0.05%), 100 μl each of a crude solution of panned poly scFV-phages, and 5, 25, 125, 625, and 3125 times dilutions were added to each well to react at room temperature for 2 hours. Each well was washed again with 0.2 ml of PBS-tween20 (0.05%) four times, a secondary antibody, that is, anti-M13-HRP (Amersham 27-9421-01) was diluted in a ratio of 1:2000, and reaction was conducted at room temperature for 1 hour. The well was washed with 0.2 ml of PBS-tween20 (0.05%), 100 μl of a substrate solution prepared by dissolving an OPD tablet (Sigmap 8787-TAB) in PC buffer [5.1 g of C₆H₈O₇.H₂O (sigma, C0706), 7.3 g of Na₂HPO₄ (sigma, S7907)] was added to each well for colorization for 10 minutes, and an optical density was measured at 490 nm using a spectrophotometer (Molecular Device, U.S.).

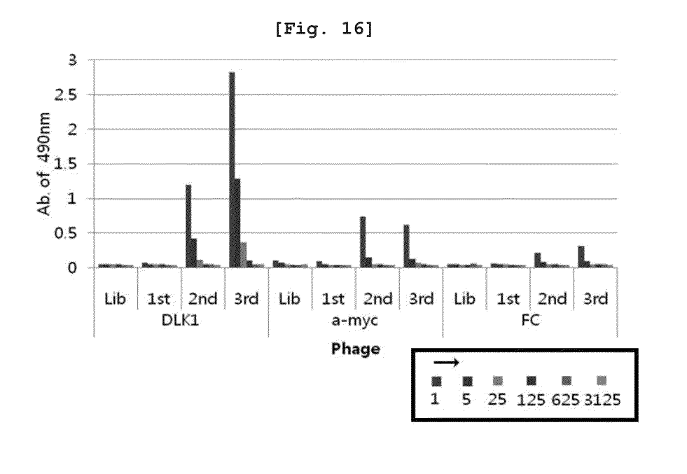

As a result, as shown in FIG. 16, it was confirmed that the bindability to an antigen increased from secondary polyclonal clone scFv-phage pools, and reached saturation at third polyclonal clone scFv-phage pools (FIG. 16).

ii) Screening of Monoclonal Antibody

Colonies obtained from a polyclone phage antibody group having high bindability were incubated in a 96-deep well plate (Bionia 90030) containing 1 ml of a medium including 2×YTCM, 2% glucose, and 5 mM MgCl₂ at 37° C. for 16 hours. 100 to 200 μl of the incubated cells were diluted in 1 ml of a medium including 2×YTCM, 2% glucose, 5 mM MgCl₂ to have a value of OD600 of 0.1, and incubated in a 96-deep well plate at 37° C. for 2 to 3 hours to have a value of OD600 of 0.5 to 0.7. An M1 helper phage was infected thereto to have a value of MOI of 1:20, and incubated in a medium including 2×YTCMK, 5 mM MgCl₂, and 1 mM IPTG at 30° C. for 16 hours. The incubated cells were centrifuged (4500 rpm, 15 minutes, 4° C.), and 4% PEG 6000 and 3% NaCl were added to and melted in a supernatant and reacted in ice for 1 hour. The resulting supernatant was centrifuged again (8000 rpm, 20 minutes, 4° C.), a pellet was melt in PBS and centrifuged (12000 rpm, 10 minutes, 4° C.), and a supernatant was transferred to a new tube and stored at 4° C. Afterward, 100 ng/well of antigens were put into a 96-well immunoplate to coat at 4° C. for 16 hours, and each well was blocked with skim milk (4%) dissolved in PBS. Each well was washed with 0.2 ml of PBS-tween20 (0.05%), and 100 μl of monoclonal scFv-phages (each 100 scFv-phage) yielded by the above method were put into each well and reacted at room temperature for 2 hours. Each well was washed again with 0.2 ml of PBS-tween20 (0.05%) four times, and a secondary antibody, that is, anti-M13-HRP, was diluted in a ratio of 1/2000, and reacted at room temperature for 1 hour. The well was washed with 0.2 ml of PBS-tween20 (0.05%) and colorized, and an optical density was measured at 490 nm. As a result, as shown in Table 2, 27 monoclonal phages having bindability to an antigen of 2 or more were screened.

TABLE 2

| | | | | | | | | | | | |
|---|---|---|---|---|---|---|---|---|---|---|---|
| 1 | 2 | 3 | 4 | 5 | 6 | 7 | 8 | 9 | 10 | 11 | 12 |

DLK1

| | | | | | | | | | | | | |
|---|---|---|---|---|---|---|---|---|---|---|---|---|
| A | 0.1129 | 0.0716 | 0.0482 | 3.1152 | 2.9859 | 0.4549 | 0.3612 | 2.92 | 0.0469 | 2.8295 | 0.2175 | 1.0026 |
| B | 0.8858 | 0.8553 | 2.0914 | 0.788 | 2.762 | 2.6351 | 2.8837 | 0.1342 | 2.3259 | 0.1396 | 2.5018 | 0.501 |
| C | 0.4976 | 0.2852 | 2.466 | 0.2239 | 0.1128 | 1.2413 | 2.9255 | 2.1548 | 0.2169 | 0.0608 | 0.2132 | 0.1591 |
| D | 0.2025 | 0.1882 | 0.1109 | 0.0586 | 0.8865 | 0.0749 | 0.0849 | 0.1145 | 0.8514 | 0.0572 | 0.1653 | 2.4751 |
| E | 0.0907 | 0.1001 | 0.0418 | 0.047 | 2.2329 | 2.3476 | 2.3778 | 0.7165 | 0.0919 | 0.7527 | 0.1737 | 0.2233 |
| F | 0.1659 | 0.2324 | 0.4055 | 2.9152 | 0.2405 | 0.933 | 0.3682 | 0.1608 | 0.2258 | 0.1668 | 2.8944 | 2.9681 |
| G | 0.0433 | 0.3815 | 0.2245 | 2.8355 | 2.5814 | 3.0216 | 0.752 | 0.3455 | 0.0609 | 0.4363 | 0.1964 | 0.0504 |
| H | 0.1044 | 2.8427 | 2.7085 | 0.296 | 0.2403 | 2.1306 | 2.8803 | 1.6389 | 3.033 | 0.5009 | 2.7793 | 3.1994 |

MYC

| | | | | | | | | | | | | |
|---|---|---|---|---|---|---|---|---|---|---|---|---|
| A | 0.2003 | 0.0474 | 0.0487 | 0.5068 | 0.7838 | 1.4922 | 0.4492 | 0.5538 | 0.0557 | 1.2823 | 0.1523 | 0.2432 |
| B | 1.357 | 0.7667 | 1.4038 | 1.1973 | 0.5932 | 0.8478 | 0.5129 | 0.1191 | 0.0918 | 0.7688 | 0.316 | 0.0526 |
| C | 0.2966 | 0.0741 | 0.2871 | 0.1538 | 0.0683 | 0.6353 | 0.5938 | 0.3595 | 0.6692 | 0.1009 | 0.1206 | 0.2206 |
| D | 0.1631 | 0.4308 | 0.078 | 0.045 | 0.5783 | 0.0632 | 0.0538 | 0.052 | 2.1443 | 0.0511 | 0.106 | 0.0889 |
| E | 0.0856 | 0.6073 | 0.0465 | 0.0435 | 0.1722 | 0.1883 | 0.4694 | 0.0867 | 0.0639 | 0.272 | 0.3112 | 0.1566 |
| F | 0.1285 | 0.4057 | 0.1421 | 1.0637 | 0.1115 | 1.1193 | 0.0898 | 1.0797 | 0.1751 | 0.1401 | 0.4419 | 0.524 |
| G | 0.0662 | 0.3396 | 0.0844 | 0.9974 | 2.9974 | 0.6732 | 0.6083 | 0.2278 | 0.0496 | 0.5198 | 0.0561 | 0.0551 |
| H | 0.0479 | 0.5654 | 1.1204 | 0.4634 | 0.066 | 0.8632 | 1.0213 | 0.6574 | 0.8562 | 0.1146 | 0.9677 | 0.6741 |

FC

| | | | | | | | | | | | | |
|---|---|---|---|---|---|---|---|---|---|---|---|---|
| A | 0.0535 | 0.0726 | 0.0731 | 0.0791 | 0.0704 | 0.1111 | 0.0748 | 0.0709 | 0.0535 | 0.0828 | 0.0591 | 0.2558 |
| B | 0.1375 | 0.4065 | 0.0851 | 0.0702 | 0.0575 | 0.0472 | 2.8291 | 0.0717 | 0.0786 | 0.0743 | 0.0548 | 0.0451 |
| C | 0.0524 | 0.0555 | 0.0521 | 0.0745 | 0.0455 | 0.0825 | 2.8824 | 0.0559 | 0.0772 | 0.0485 | 0.0663 | 0.061 |
| D | 0.0519 | 0.0686 | 0.0447 | 0.0722 | 0.0431 | 0.0455 | 0.0482 | 0.0528 | 0.0498 | 0.1141 | 0.0651 | 0.0831 |
| E | 0.0496 | 0.0543 | 0.0419 | 0.0587 | 0.0472 | 0.0481 | 0.0558 | 0.2673 | 0.0492 | 0.1508 | 0.0601 | 0.0577 |
| F | 0.0614 | 0.0584 | 0.0528 | 0.0879 | 0.0553 | 0.1223 | 0.0792 | 0.0756 | 0.0661 | 0.0922 | 0.0658 | 0.0757 |
| G | 0.0456 | 0.0687 | 0.0521 | 0.1171 | 2.079 | 0.0689 | 0.547 | 0.0991 | 0.0874 | 0.2255 | 0.1894 | 0.0457 |
| H | 0.0594 | 0.0642 | 0.049 | 0.0705 | 0.0608 | 0.0766 | 0.1107 | 0.0819 | 0.0701 | 0.1204 | 0.0592 | 0.0539 |

(3) Classification and Examination of Monoclonal Phages

i) Verification by Finger Printing

0.2 μl of a Taq.DNA polymerase (Gendocs, 5 U/μl), 50 p/μl of a forward primer (SEQ. ID. NO: 52: 5′-CTAGATAACGAGGGCAAATCATG-3′), 0.2 μl of a reverse primer (SEQ. ID. NO: 53: 5′-CGTCACCAATGAAACCATC-3′), 3 μl of 10× buffer, 0.6 μl of 10 mM dNTP mix, and 24.8 μl of distilled water were mixed with 1 μl of 27 monoclonal cells for primarily-screened DLK1-Fc, and colony PCR (iCycler iQ, BIO-RAD) was performed. Conditions for the PCR program are shown in Table 3.

TABLE 3

| | | |
|---|---|---|
| Temperature | Time | Cycle |

| | | | |
|---|---|---|---|
| 95° C. | 5 | min | |
| 95° C. | 30 | sec | 30 cycles |
| 56° C. | 30 | sec | |
| 72° C. | 1 | min | |
| 72° C. | 10 | min | |
| 4° C. | | | |

The colony PCR product was detected in 1% agarose gel (Seakem LE, CAMERES 50004), 0.2 μl of BstNI (Roche11288075001, 10 U/μl) was added to react at 37° C. for 2 to 3 hours. Reaction conditions are shown in Table 4. The digested product was detected in 8% DNA polyacryl amide gel.

TABLE 4

| | |
|---|---|
| 10x Buffer | 3 μl |
| Colony PCR product | 10 μl |
| BstNI (10 U/μl) | 0.2 μl |
| Distilled water | 16.8 μl |

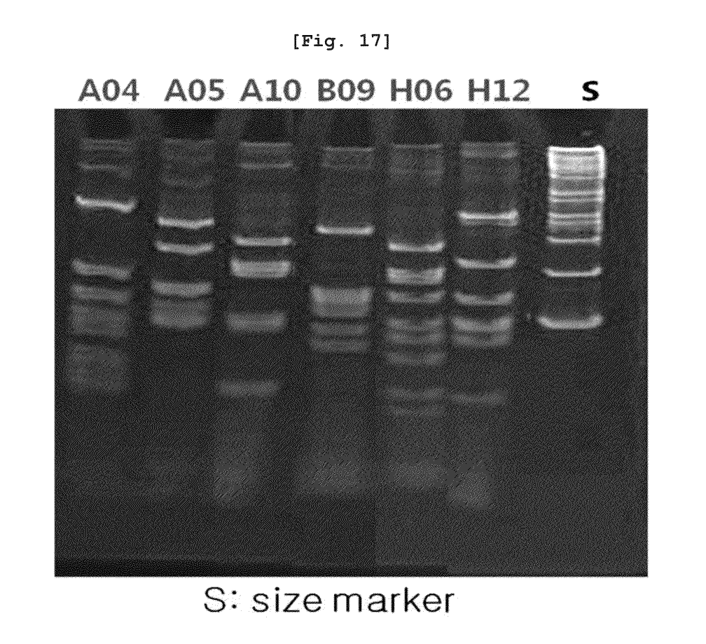

As a result, as shown in FIG. 17, diversity of fragments of monoclonal phage antibodies digested with BstNI is identified, and it is estimated that 6 kinds of different antibodies were screened.

ii) Verification by Sequencing

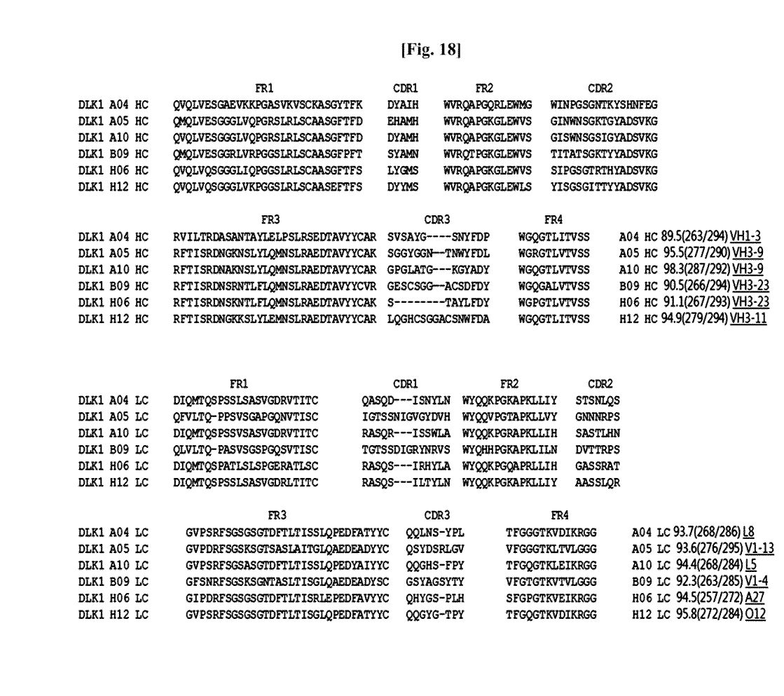

6 kinds of monoclonal phages with respect to water-soluble DLK1 were incubated in a medium including 2×YTCM, 2% glucose, and 5 mM MgCl₂ (5 ml) at 37° C. for 16 hours. DNAs were harvested from the incubated monoclones using a DNA purification kit (Nuclogen 5112), and subjected to sequencing using a primer set forth in SEQ. ID. NO: 52 (SolGent, Korea). As a result, as shown in Table 5 and FIG. 18, CDRs of VH and VL of the screened antibody were identified.

TABLE 5

| Clone | | Identi- | CDR3-a.a. | | Identi- | CDR3-a.a | | | | | |
|---|---|---|---|---|---|---|---|---|---|---|---|
| Name | VH | ties | seq | VL | ties | seq | Group | DLK1 | a-myc | Fc | Ratio |
| DLK1A04 | VH1-3 | 263/294 | SVSAYG---- | L8 | 268/286 | QQLNS- | 1 | 3.1152 | 0.5068 | 0.0791 | 6.146803473 |
| | | (89.5%) | SNYFDP | | (93.7%) | YPL | | | | | |
| DLK1A05 | VH3-9 | 277/290 | SGGYGGN-- | V1-13 | 276/295 | QSYDSR | 2 | 2.9859 | 0.7838 | 0.0704 | 3.809517734 |
| | | (95.5%) | TNWYFDL | | (93.6%) | LGV | | | | | |
| DLK1A10 | VH3-9 | 287/292 | GPGLATG--- | L | 268/284 | QQGHS- | 3 | 2.8295 | 1.2823 | 0.0828 | 2.206581923 |
| | | (98.3%) | KGYADY | | (94.4%) | FPY | | | | | |
| DLK1B09 | VH3-23 | 266/294 | GESCSGG-- | V1-4 | 263/285 | GSYAGS | 4 | 2.3259 | 0.0918 | 0.0786 | 25.33660131 |
| | | (90.5%) | ACSDFDY | | (92.3%) | YTY | | | | | |
| DLK1H06 | VH3-23 | 267/293 | S-------- | A27 | 257/272 | QHYGS- | 5 | 2.1306 | 0.8632 | 0.0766 | 2.468257646 |
| | | (91.1%) | TAYLFDY | | (94.5%) | PLH | | | | | |
| DLK1H12 | VH3-11 | 279/294 | LQGHCSGGAC | O12 | 272/284 | QQGYG- | 6 | 3.1994 | 0.6741 | 0.0539 | 4.746180092 |
| | | (94.9%) | SNWFDA | | (95.8%) | TPY | | | | | |

The similarity between these antibodies and a germ cell line antibody group was estimated using an Ig BLAST program (www.ncbi.nlm.nih.gov/igblast/) of NCBI, and therefore 6 kinds of phage antibodies specific to water-soluble DLK1 were obtained. Here, in a heavy chain, 2 kinds each of VH3-9 and VH3-23, and one kind each of VH3-11 and VH1-3 were included. Amino acid sequences of CDR3 of a heavy chain and a light chain of the antibody were analyzed, and it was confirmed that they had different sequences from each other (FIG. 18).

(4) Analysis of Characteristics of Human Antibody with Respect to Water-Soluble DLK1

i) Analysis of Total IgG Conversion

To convert monoclonal phage antibodies to water-soluble DLK1 into an IgG total vector in a phage, a heavy chain was subjected to colony PCR (iCycler iQ, BIO-RAD) by mixing 1 μl of monoclonal DNA, 10 pmole/μl of forward and reverse primers for a heavy chain shown in Table 6, 5 μl of 10× buffer, 1 μl of a 10 mM dNTP mix, 0.5 μl of a pfu DNA polymerase (SolGent, 2.5 U/μl) and distilled water. A light chain was also subjected to colony PCR using forward and reverse primers for a light chain shown in Table 6.

TABLE 6

| Name | | | | |
|---|---|---|---|---|
| of | HC | | | |
| clone | Forward primer | | Reverse primer | |
| A04 HC | NATVH3-2 | SEQ. ID. NO: 5 | NATJH- | SEQ. ID. NO: 8 |
| A05 HC | NATVH7-1 | SEQ. ID. NO: 6 | ALL | |
| A10 HC | NATVH3-2 | SEQ. ID. NO: 5 | | |
| B09 HC | NATVH7-1 | SEQ. ID. NO: 6 | | |
| H06 HC | NATVH1-1 | SEQ. ID. NO: 7 | | |
| H12 HC | | | | |

| Name | | | | |
|---|---|---|---|---|
| of | LC | | | |
| clone | Forward primer | | Reverse primer | |
| A04 HC | NATVK6 | SEQ. ID. NO: 9 | NATJK-R7 | SEQ. ID. NO: 12 |
| A05 HC | NATVL13 | SEQ. ID. NO: 10 | NATJL2-R | SEQ. ID. NO: 13 |
| A10 HC | NATVK6 | SEQ. ID. NO: 9 | NATJK-R5 | SEQ. ID. NO: 14 |
| B09 HC | NATVL10 | SEQ. ID. NO: 11 | NATJL1-R | SEQ. ID. NO: 15 |
| H06 HC | NATVK6 | SEQ. ID. NO: 9 | NATJK-R4 | SEQ. ID. NO: 16 |
| H12 HC | | | NATJK-R7 | SEQ. ID. NO: 12 |

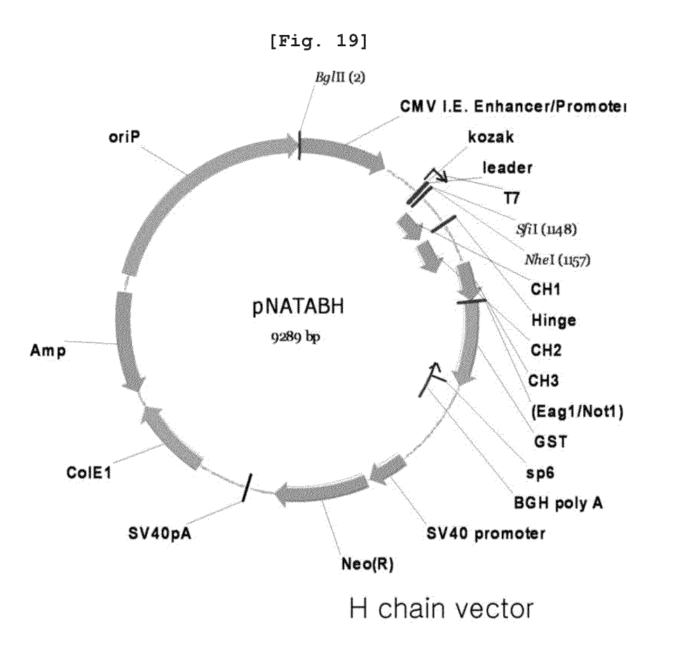

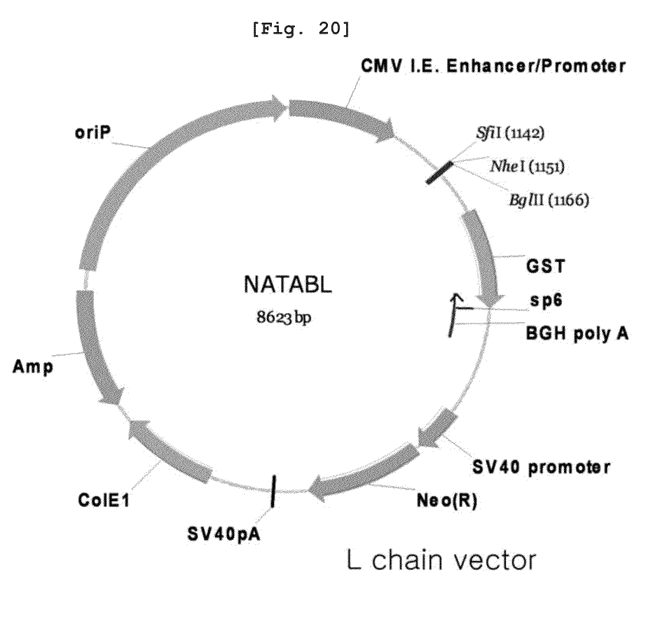

A heavy chain gene yielded by PCR was purified using a DNA-gel extraction kit (Qiagen), 1 μl (10 ng) of pNATAB H vector (FIG. 19), 15 μl of a heavy chain (100˜200 ng), 2 μl of 10× buffer, 1 μl of a ligase (1 U/μl), and distilled water were added to the gene, and left at room temperature for 1 to 2 hours to be linked to the vector. The vector was left with cells for transformation (XL1-blue) in ice for 30 minutes, and shocked by heat at 42° C. for 90 seconds, thereby performing transduction. The transduced product was left in ice for 5 minutes, 1 ml of an LB medium was injected to incubate at 37° C. for 1 hour. Afterward, the incubated cells were plated on an LB Amp solid medium, and incubated at 37° C. for 16 hours. A single colony was inoculated into 5 ml of a LB Amp liquid medium, and incubated at 37° C. for 16 hours. From the above culture solution, DNA was extracted using a DNA-prep. kit (Nuclogen). In addition, from a light chain, DNA was extracted by the same method as described above using a pNATAB L vector (FIG. 20).

The yielded DNA was subjected to sequencing using a CMV-proF primer (SEQ. ID. NO: 54: AAA TGG GCG GTA GGC GTG; SolGent). As a result, it was confirmed that sequences of heavy chains and light chains of 6 clone phages with respect to DLK1-Fc converted into total IgG corresponded to a sequence of a phage antibody.

12-2. Verification of Human Antibody Specifically Binding to Water-Soluble Domain of DLK1

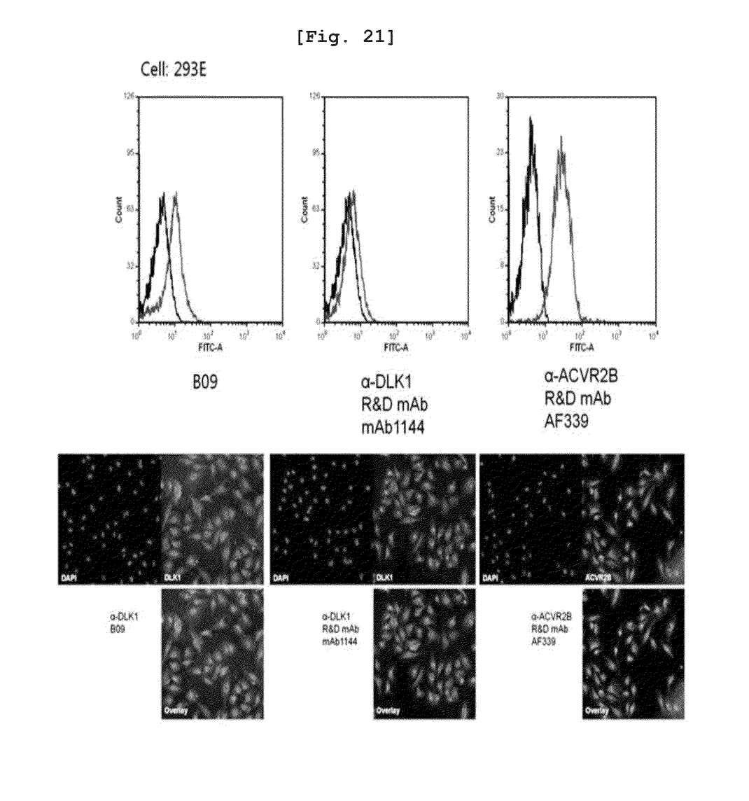

To identify a binding strength of a human antibody specifically binding to a water-soluble domain of DLK1 manufactured in Example 12-1, representatively, flow cytometry migration analysis was performed using B09 antibody of 6 kinds of human antibodies and 293E cells expressing DLK1, and results are shown in FIG. 21. Here, a binding strength between a DLK1 monoclonal antibody purchased from R&D and ACVR2B antibody was measured.

As a result, as shown in FIG. 21, it was confirmed that B09 antibody specifically bound to a water-soluble domain of DLK1. In addition, it was confirmed that the DLK1 monoclonal antibody purchased from R&D and ACVR2B antibody also bound to DLK1 and ACVR2B, respectively (FIG. 21).

### Example 13

### Measurement of Binding Strength by Surface Plasmon Resonance

To measure a binding strength, surface plasmon resonance was performed using ProteOn XPR36 produced by Bio-Rad, and the method is summarized as follows. As 0.1 M EDC and 0.025 M sulfo-NHS flew for 60 seconds to activate a sensor chip, a protein to be coated was put into 10 mM sodium acetate (pH 5.0), and flew for 240 seconds at 30 μl/min to perform attachment to the chip. A channel was blocked for 200 seconds with 1 M ethanolamine-HCl (pH 8.5), and washed with DPBST (PBS with 0.005% tween 20). To estimate a binding strength, the resulting product was two times serial diluted and flew at 30 μl/min for 120 minutes to measure the binding strength. To estimate dissociation strength, a dissociation phase was given for 240 seconds.

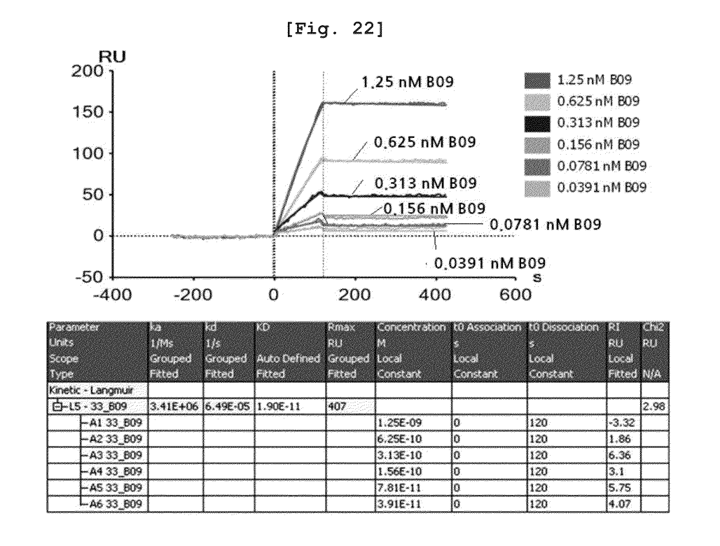

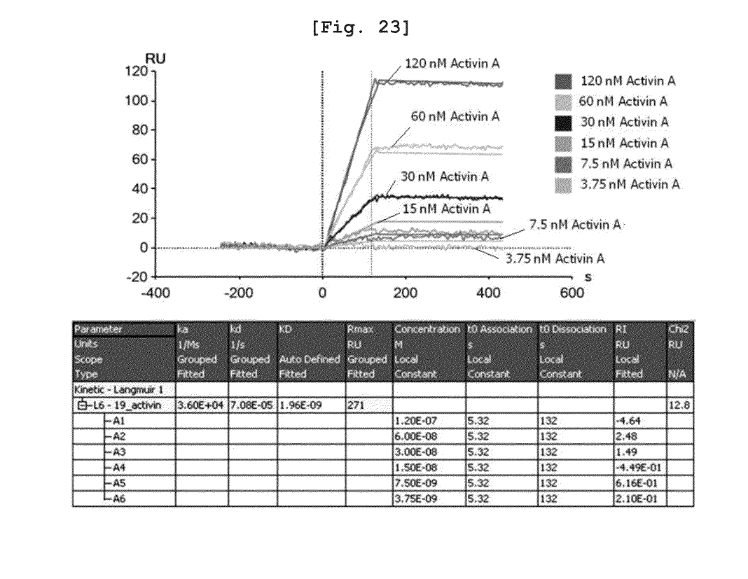

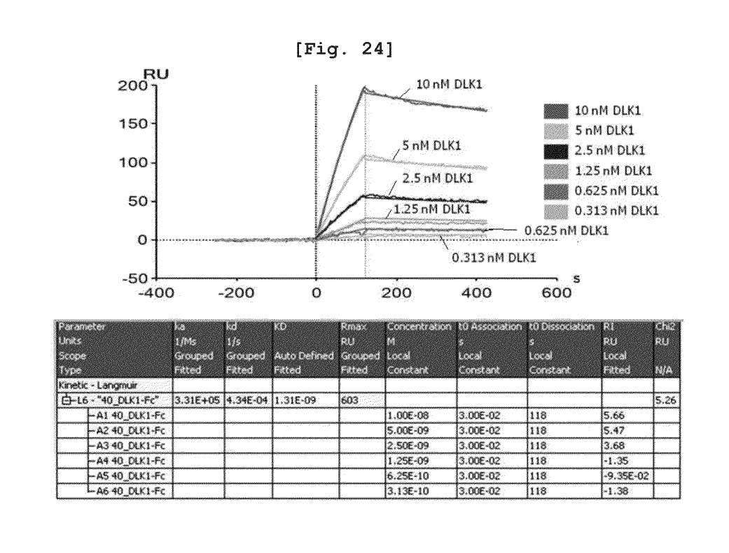

As shown in FIG. 22, the binding strength of the B09 antibody to water-soluble DLK1 was measured at a Kd value of 1.90E-11 (FIG. 22). In addition, binding strength of activin A to ACVR2B was measured at a Kd value of 1.96E-09 (FIG. 23), and binding strength of water-soluble DLK1 to ACVR2B was measured at a Kd value of 1.31E-09 (FIG. 24).

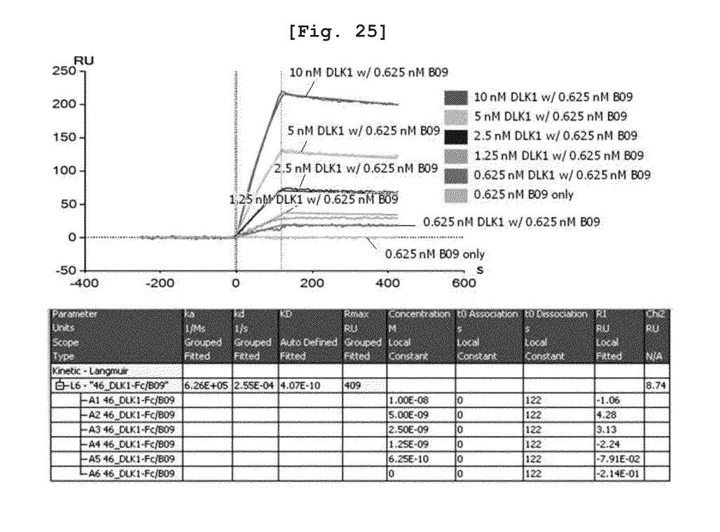

In addition, to quantitatively analyze an effect of water-soluble DLK1 on the binding between activin A and ACVR2B, binding strength for each was measured. As a result, when B09 antibody was treated in the binding between water-soluble DLK1 and ACVR2B, the binding strength is increased and measured at 4.07E-10 (FIG. 25).

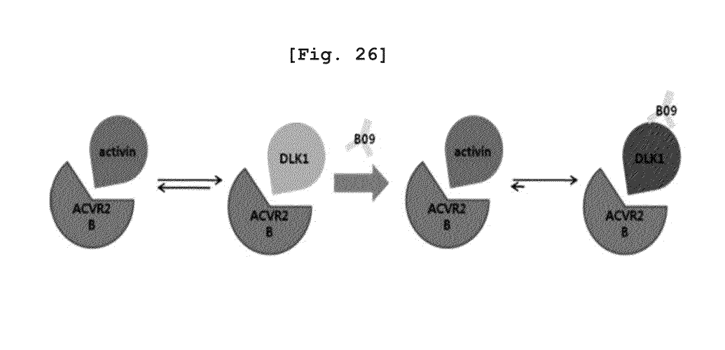

The results are summarized and shown in FIG. 26 showing that B09 antibody specifically binding to a water-soluble domain of DLK1 was an agonistic antibody exhibiting a synergetic action, and comparing a binding coefficient with a dissociation coefficient, when water-soluble DLK1 and ACVR2B were bound to each other, a more stable structure was formed and thus a dissociation rate was decreased.

### Example 14

### Enzyme Immunoassay (ELISA)

To estimate an effect of an antibody on the binding between water-soluble DLK1 and ACVR2B or the binding between activin A and ACVR2B, an enzyme immunoassay was performed. 50 ng/ml of ACVR2B-Fc or 200 ng/ml of activin A was coated, and reacted with FLAG-DLK1, ACVR2B-Fc or DLK1-Fc, and antibodies thereof were added to observe competitive binding.

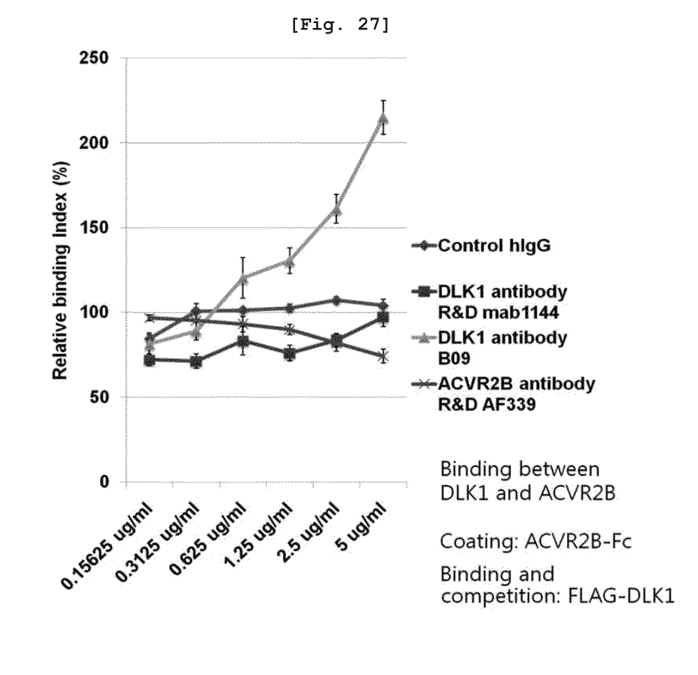

First, according to that effects of the antibodies on the binding between water-soluble DLK1 and ACVR2B was examined by coating ACVR2B, binding water-soluble DLK1, and treating respective antibodies, it was confirmed that the B09 antibody exponentially increased the binding strength of water-soluble DLK1 to ACVR2B, and as R&D AF339 antibody which was known to have a neutralizing effect on the binding between activin A and ACVR2B was treated, the binding between water-soluble DLK1 and ACVR2B was slightly reduced (FIG. 27). Such a result showed that B09 antibody could significantly increase the binding between water-soluble DLK1 and ACVR2B and the binding is decreased by AF339 antibody, and thus it is estimated that a part involved with the binding between activin A and ACVR2B partially overlapped a part involved with the binding between water-soluble DLK1 and ACVR2B.

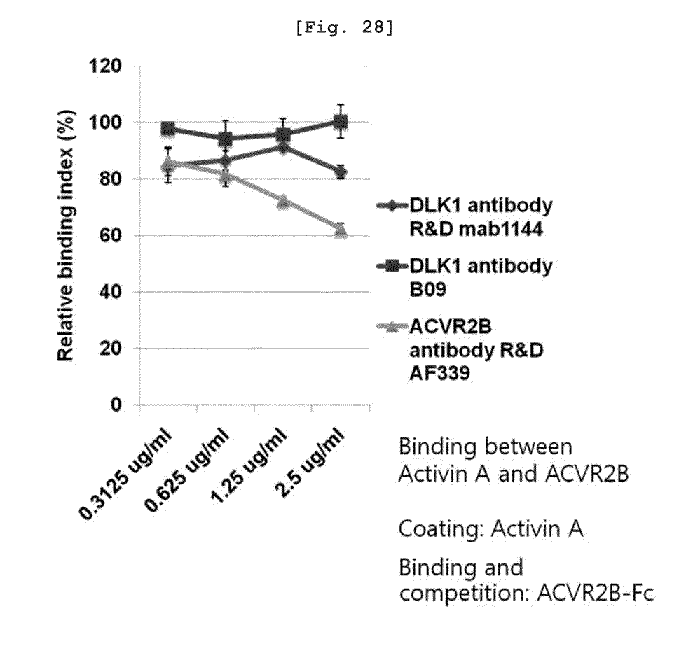

Subsequently, according to that an effect on the binding between activin A and ACVR2B by coating activin A, binding ACVR2B-Fc, and treating an antibody for each, it was confirmed that the binding between activin A and ACVR2B was decreased by treatment of R&D AF339 antibody, and DLK1 antibodies was not directly involved with the binding between activin A and ACVR2B (FIG. 28).

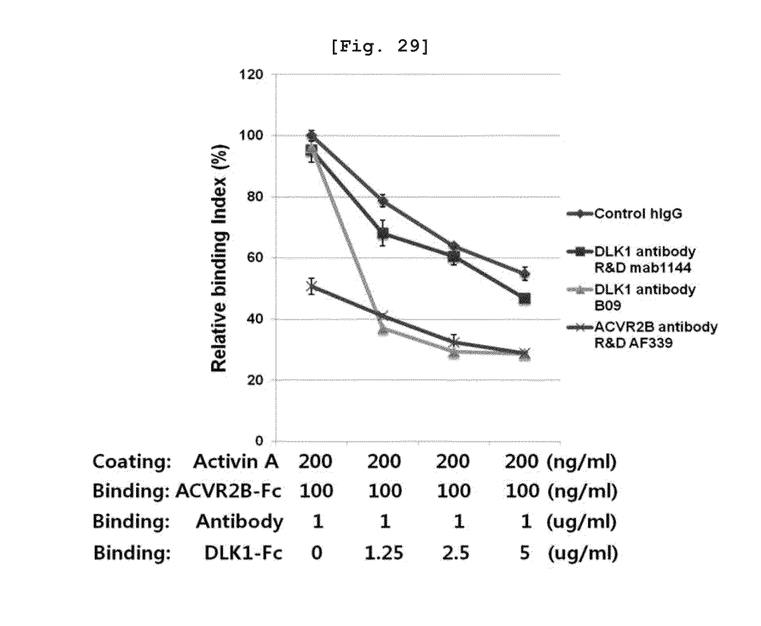

In addition, effects of water-soluble DLK1, DLK1 antibody and ACVR2B antibody on the binding between activin A and ACVR2B was examined by coating activin A, binding ACVR2B-Fc, and treating water-soluble DLK1 and an antibody for each. As a result, it was confirmed that, as concentration of water-soluble DLK1 was increased, binding strength between activin A and ACVR2B was decreased, and when B09 antibody was also treated, the binding strength between activin A and ACVR2B was significantly decreased. In addition, it was seen that an inhibitory effect of water-soluble DLK1 on the binding between activin A and ACVR2B was more increased by treatment of R&D AF339 antibody (FIG. 29). Such a result shows that the best effect could be obtained when B09 antibody having a synergenic action with water-soluble DLK1 or an antibody suppressing the binding between ACVR2B and activin A was treated together to inhibit activin signal transduction.

### Example 15

### Western Blotting for Verifying Effect of Water-Soluble DLK1 on Activin Signal Transduction

When a pancreatic cancer cell line, MIA-PaCa-2, was grown approximately 70% in a culture container, a medium was transferred with a serum-free medium, and the cells were incubated for 16 hours. 10 μg/ml of DLK1-Fc was treated to a DLK1-Fc treated group, 0.5 nM activin A was added, and then the cells were harvested after 30 minutes. The harvested cells were lysed using a buffer prepared by adding a protease inhibitor (Roche), and phosphotase inhibitor cocktail I and II (Sigma) to RIPA buffer (50 mM TrisHCl pH 7.4, 150 mM NaCl, 2 mM EDTA, 1% NP-40), and a concentration of the cell lysate was determined using a BCA quantification kit (Thermo). 30 μg of the lysate was denatured using a 5× sample buffer in SDS-PAGE, subjected to electrophoresis, and transferred to a PVDF membrane (GE). Obtained blots were subjected to western blotting using psamd2/3, Smad2/3, Smad4(Cell signaling), and Smad7(Milipore). Here, a beta-actin antibody (Sigma) was used as a loading control. After the reaction was done overnight at 4° C., the cells were washed with TBST (Tween 20 0.05%) three times, reacted with a rabbit-HRP antibody and a mouse-HRP antibody (Santa Cruz) for 1 hour at room temperature, and washed again with TBST (Tween 20 0.05%) three times. Afterward, the blots were soaked in an ECL solution (Intron) and exposed to a film.

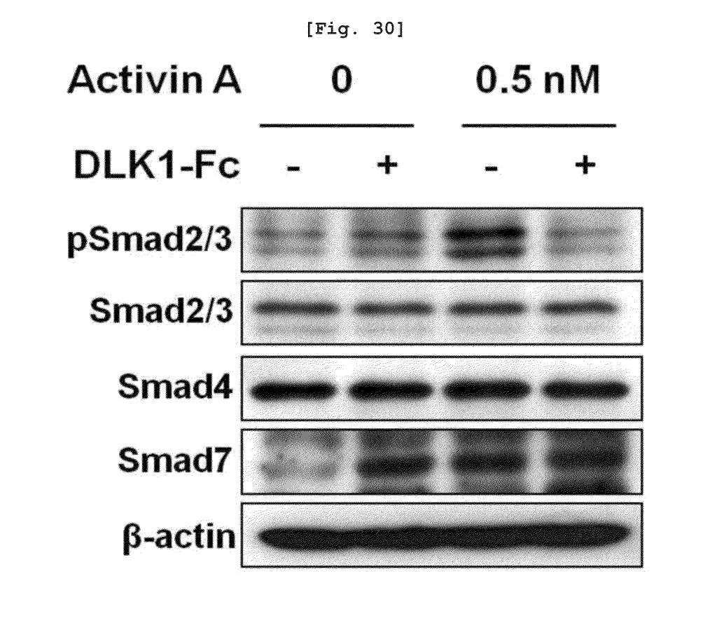

As a result, as shown in FIG. 30, it was seen that activin signal transduction did not occur due to the decrease in pSmad2/3, and Smad7 serving to decrease the activin signal transduction was increased (FIG. 30).

### Example 16

### Effect of Water-Soluble DLK1 on Smad Signal Transduction

To examine the effect of water-soluble DLK1 on Smad signal transduction, SEAP reporter analysis was performed. Since a vector used in the SEAP reporter analysis had a Smad binding element (SBE) increased by the activin signal transduction prior to a SEAP gene, when transcriptional regulation by Smad occurred in cells, the vector had a system expressing a reporter gene. The vector was introduced to a MIA-PaCa-2 cell line by electrophoresis. Next day, the transfected cell line was transferred to a 96-well plate, incubated for 24 hours, and further incubated with a serum-free medium for 16 hours. Fc or DLK1-Fc was pre-treated to the cells for 1 hour, 0.5 nM activin A was treated, and the resulting cells were incubated for 48 hours. The cell culture solution was taken to perform a reporter analysis. The analysis was performed using a Phospha-Light system produced by ABI. In addition, the same experiment as described above was performed using a B09 antibody.

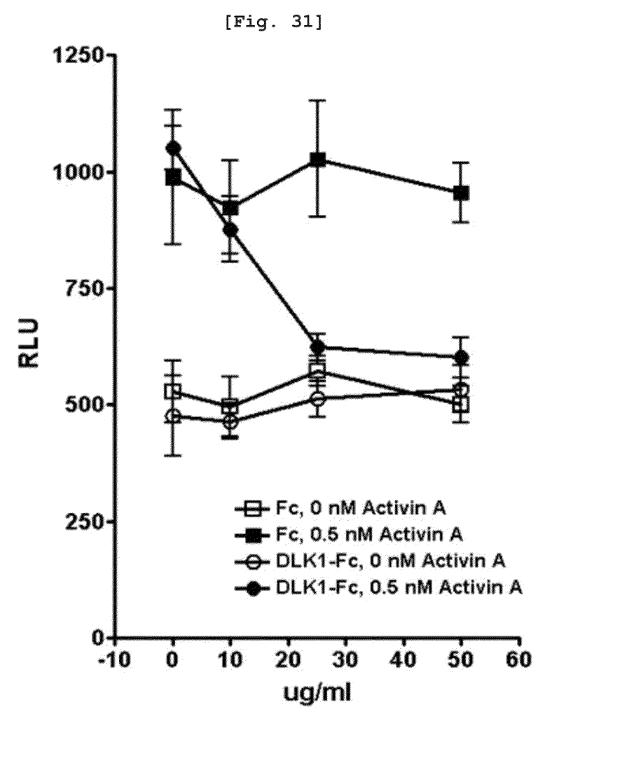

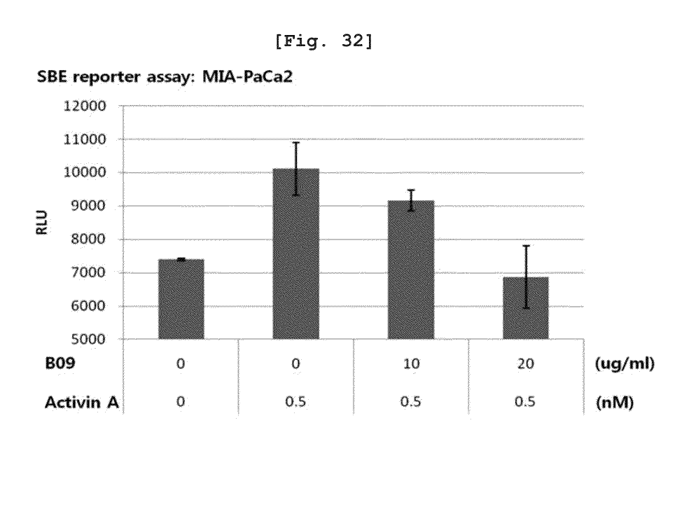

As a result, it was seen that a signal of the reporter system was decreased by the treatment of DLK1-Fc (FIG. 31), and activin signal transduction was decreased according to an amount of the B09 antibody added (FIG. 32).

### Example 17

### Effect of B09 Antibody on Hypoxia Signal Transduction

A reporter analysis was performed to examine an effect of B09 antibody specifically binding to a water-soluble domain of DLK1 on signaling under a hypoxia condition in which DLK1 was overexpressed. Since a vector used in the reporter analysis had a hypoxia response element (HRE) prior to a luciferase gene, when transcriptional regulation occurred in cells due to hypoxia, the vector had a system expressing a reporter gene. The vector was introduced to a MIA-PaCa-2 cell line by electrophoresis (Invitrogen). Next day, the transfected cell line was transferred to a 96-well plate, incubated for 24 hours, and further incubated with a serum-free medium for 16 hours. During the incubation with the serum-free medium, 100 nM CoCl₂ (Sigma) was treated, thereby inducing artificial hypoxia. The cells were pre-treated with a DLK1 antibody for 1 hour, and a reporter analysis was performed. The analysis was performed using a Phospha-Light system produced by ABI.

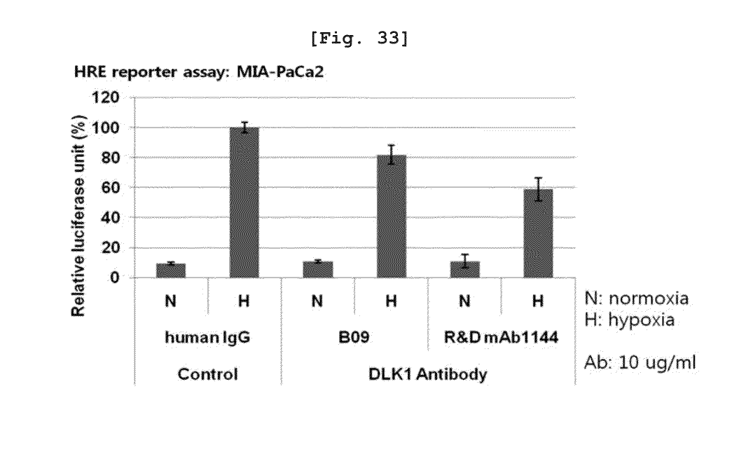

As a result, as shown in FIG. 33, it was seen that the B09 antibody could inhibit hypoxia signal transduction (FIG. 33).

### Example 18

### Confirmation of Effects of ACVR2B Antibody and DLK1 Antibody on an Inhibitory Effect of DLK1 Antibody on Cancer Migration and Inhibitory Effect of Water-Soluble DLK1 on Cancer Migration

To examine an inhibitory effect of a DLK1 antibody on cancer migration, and effects of an ACVR2B antibody and a DLK1 antibody on an inhibitory effect of water-soluble DLK1 on cancer migration, a migration experiment was performed using a MIA-PaCa-2 cell line. First, when cells were grown approximately 70%, a medium was transferred with a serum-free medium, after 24 hours, the cells were dissociated by trypsine treatment, and a cell count was measured. The cells, the serum-free medium, and each protein to be treated were mixed to have a total volume of 100 μl, and incubated at 37° C. for 1 hour. 1 ml of 5˜10% FBS was added to a 24-well plate as a chemo-attractant, a transwell (Corning #3422) having a 8.0 μm pore was put thereon, DLK1 and an antibody were added thereto with pre-incubated cells and 100 μl of a mixed solution of the cells and proteins for 1 hour, and the resulting solution was incubated in a carbon dioxide incubator at 37° C. for 24 to 48 hours. After the incubation, the medium in the transwell was removed, the cells were dyed using a Diff Quick solution (Sysmex, Japan), and cells not passing through the transwell were completely removed with a swab. Afterward, the transwell was completely dried, cells passing through the transwell were observed with 400 magnification and photographed.

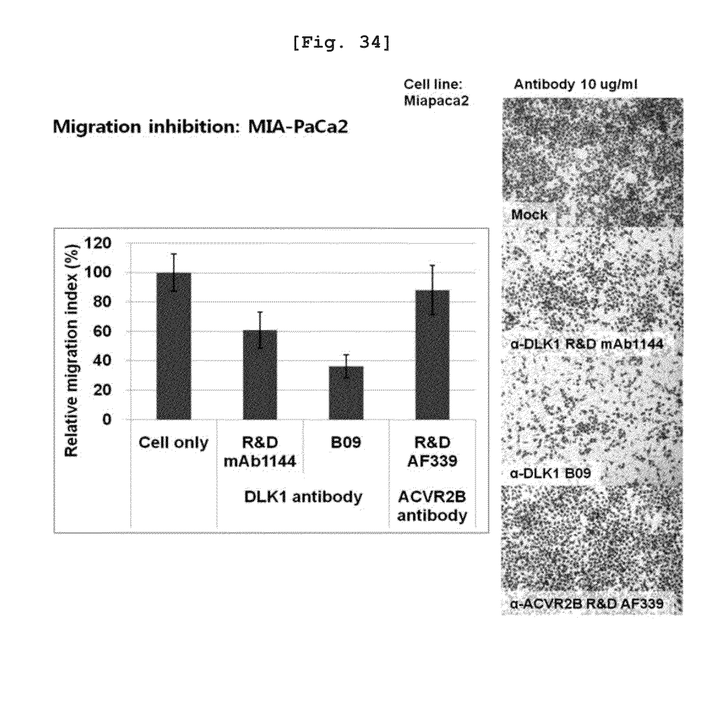

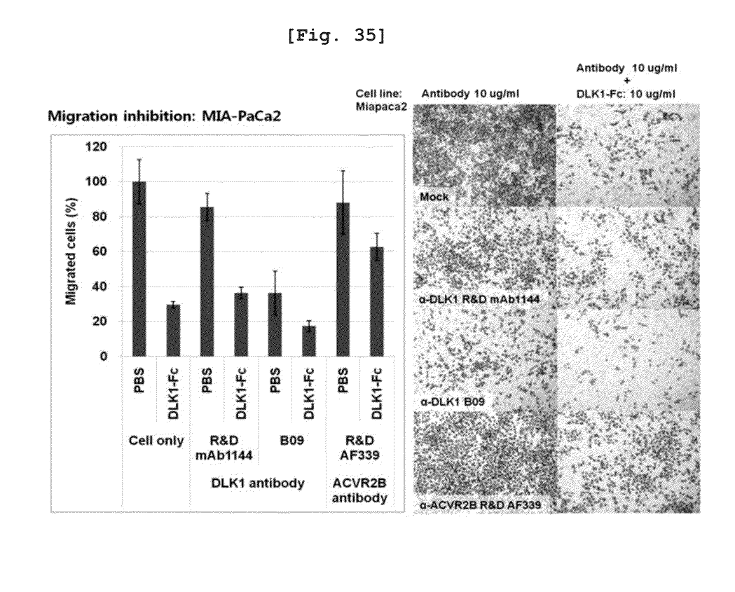

As a result, it was confirmed that the DLK1 antibody showed better migration suppression than the ACVR2B antibody (FIG. 34). In addition, according to the effects on migration when water-soluble DLK1 was treated together, when a mab1144 antibody was treated in combination with the water-soluble DLK1, an effect on migration suppression was not significant, but when a B09 antibody is treated alone or in combination with the water-soluble DLK1, excellent migration suppression was shown. It was also confirmed that when an AF339 antibody was treated, an effect of the water-soluble DLK1 on migration suppression was decreased (FIG. 35).

### Example 19

### Orthotopic Animal Model with MIA-Paca-2 Pancreatic Cancer

To confirm an effect of DLK1-Fc on cancer migration and suppression of proliferation of cancer cells, an experiment for an orthotopic animal model with MIA-Paca-2 pancreatic cancer was performed. Nude mice used in the experiment were 6 week-old females (Can Cg-Foxnl-nu/CrljBgi), which were purchased from Orient Bio Inc. The mice were adapted for approximately 1 week, and 5×10⁶ of pancreatic cancer cell lines, MIA-Paca-2, were orthotopic-transplanted to the pancreas of a mouse. The cell lines were divided into two groups, and in one group, 5 mpk of DLK1-Fc was injected, and in the other group, 15 mpk of DLK1-Fc was injected. PBS was used as a control. Proteins were injected by intraperitoneal injection twice a week. After 5 weeks of the orthotopic transplantation, a result of the experiment was observed through dissection.

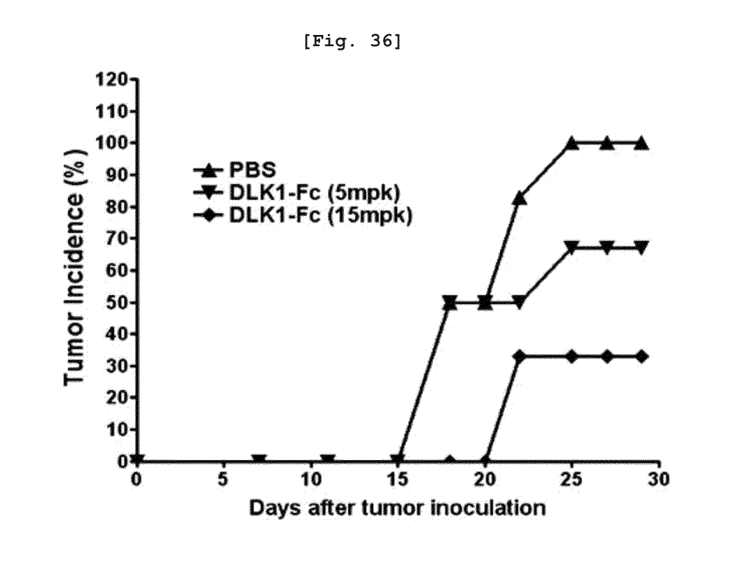

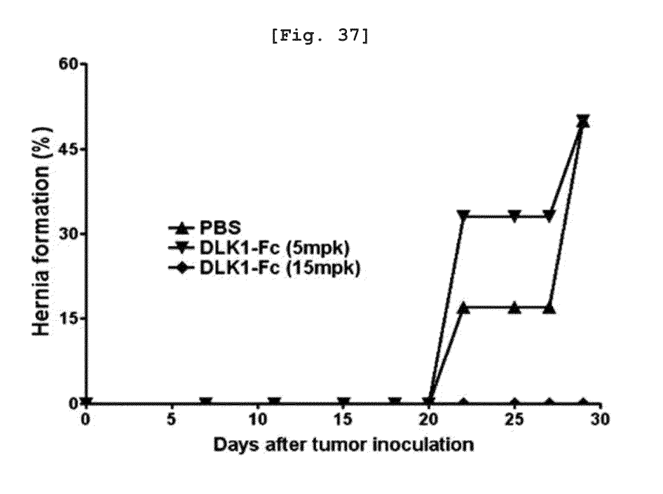

Before dissection, a ratio of tumor occurrence was examined, from which it was seen that the ratio of tumor occurrence was significantly decreased in a DLK1-Fc treated group (FIG. 36), and a ratio of hernia formation was examined by the dissection, from which the ratio of hernia formation was significantly decreased in the DLK1-Fc treated group (FIG. 37).

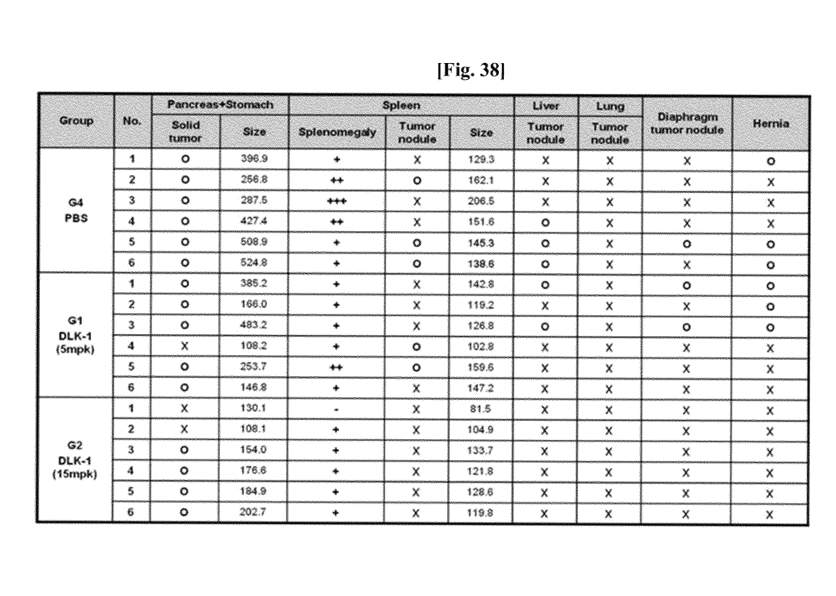

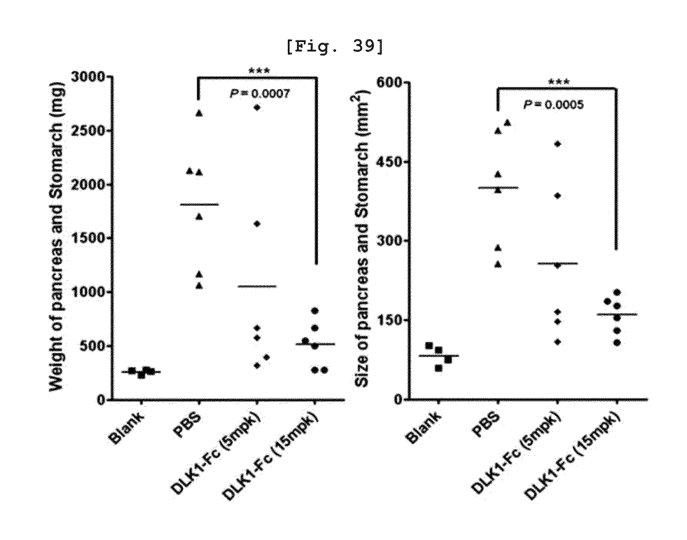

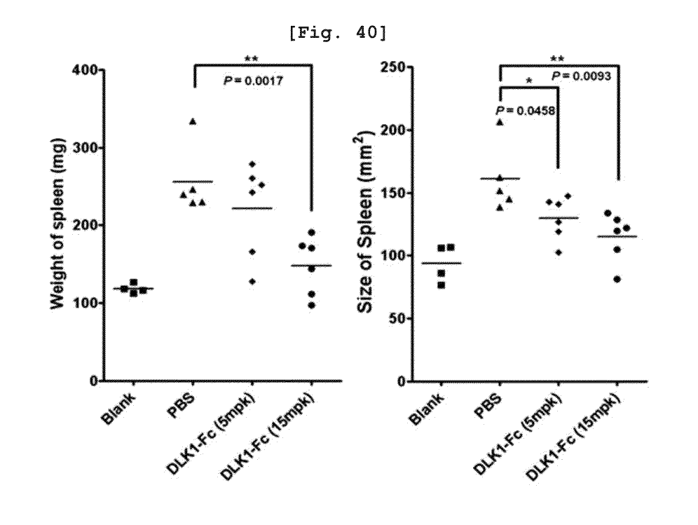

In addition, it was seen that migration to liver, lung, and diaphragm was significantly decreased, and thus proliferation of cancer cells was reduced in the DLK1-Fc treated group (FIG. 38), and according to the result obtained by analyses of sizes and weights of the pancreas and lung after dissection, it was confirmed that formation of cancer was significantly suppressed in the DLK1-Fc treated group (FIG. 39). In addition, like the result of the measurement of enlargement of a spleen by a tumor, compared to a control, it was confirmed that the enlargement of a spleen was significantly decreased in the DLK1-Fc treated group (FIG. 40).

### Example 20

### Small Animal In Vivo Imaging (SAIVI)

To examine distribution of injected DLK1-Fc in an actual mouse, fluorescent quantum dots were labeled to DLK1-Fc to perform a SAIVI experiment. Nude mice used in the experiment were 6 week-old females (Can Cg-Foxnl-nu/CrljBgi), which were purchased from Orient Bio Inc. The labeled quantum dot was QDs 800 (Invitrogen), and as a control, only QDs 800 was used. First, 5×10⁶ of pancreatic cancer cell lines, MIA-Paca-2, were orthotopic-transplanted to the pancreas of a mouse. After a cancer tissue was formed, the quantum dots were injected by intravenous injection, and fluorescence was detected, thereby performing in vivo imaging. For imaging, a Maestro in vivo imaging system (CRi, Inc.) was used.

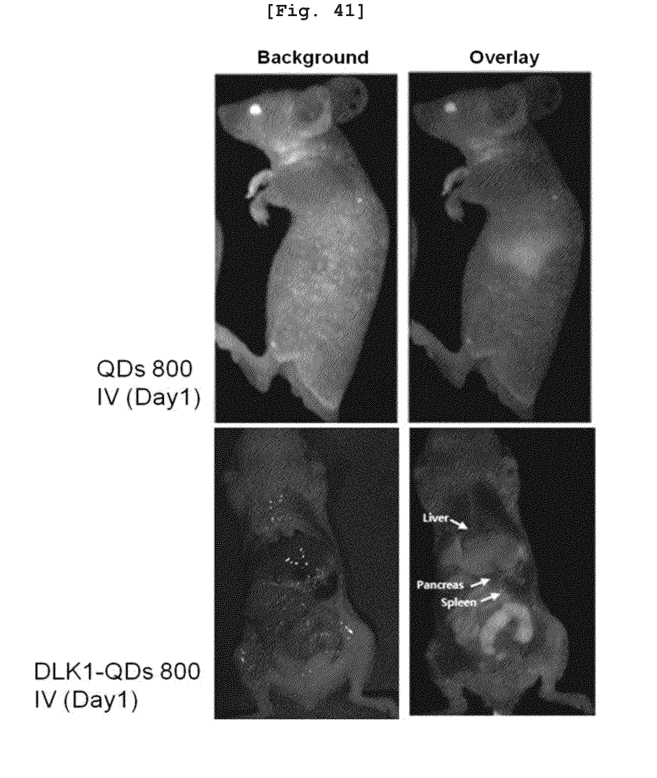

As a result, it could be confirmed that the treated DLK1-Fc was collected at the place of cancer cells (FIG. 41). It is considered that this result was because ACVR2B was more overly expressed in the cancer cells than cells in normal tissues.

As protein signal transduction involved with a ligand is suppressed, a composition of the present invention can be useful to prevent or treat relating diseases.

1. A method of suppressing binding between an activin receptor type II B (ACVR2B) and a ligand having ACVR2B as a receptor, comprising a step of administering to a subject an effective amount of a composition comprising:
an extracellular water-soluble domain of delta-like 1 homolog (DLK1), a fragment of the extracellular water-soluble domain of DLK1, a mutant of the extracellular water-soluble domain of DLK1 or a fragment of the mutant as an active ingredient.

2. The method according to claim 1, wherein the extracellular water-soluble domain of DLK1 comprises an amino acid sequence set forth in SEQ. ID. NO: 1.

3. The method according to claim 1, wherein the ligand having ACVR2B as a receptor is selected from the group consisting of activin, Nodal, bone morphogenetic protein 6 (BMP6), bone morphogenetic protein 7 (BMP7), growth/differentiation factor 5 (GDF5), and growth/differentiation factor 11 (GDF 11).

4. The method according to claim 1, wherein the extracellular water-soluble domain of DLK1, the fragment of the extracellular water-soluble domain of DLK1, the mutant of the extracellular water-soluble domain of DLK1 or the fragment of the mutant competitively binds to ACVR2B with the ligand having ACVR2B as a receptor.

5. The method according to claim 1, wherein the composition further comprises a fragment including a human antibody specifically binding to the extracellular water-soluble domain of DLK1, or an antigen binding site thereof.

6. A method of suppressing binding between ACVR2B and a ligand having ACVR2B as a receptor, comprising a step of administering to a subject an effective amount of a composition comprising:
an extracellular water-soluble domain of DLK1 or a fragment thereof, and a DLK1-Fc fusion protein to which an Fc region of a human antibody is linked as active ingredients.

7. The method according to claim 6, wherein the ligand having ACVR2B as a receptor is selected from the group consisting of activin, Nodal, BMP6, BMP7, GDF5, and GDF11.

8. A method of preventing or treating a disease selected from the group consisting of cancer, a metabolic disease, an immunological disorder, and a liver disease, in a subject in need, the method comprising the step of administering to the subject an effective amount of a pharmaceutical composition, wherein the pharmaceutical composition comprises at least one selected from the group consisting of:
(a) an extracellular water-soluble domain of delta-like 1 homolog (DLK1), a fragment of the extracellular water-soluble domain of DLK1, a mutant of the extracellular water-soluble domain of DLK1, or a fragment of the mutant as an active ingredient; and
(b) an extracellular water-soluble domain of DLK1 or a fragment thereof, and a DLK1-Fc fusion protein to which an Fc region of a human antibody is linked as an active ingredient.

9. The method according to claim 8, wherein the extracellular water-soluble domain of DLK1 competitively binds to ACVR2B with activin.

10. The method according to claim 8, wherein the composition has an inhibitory effect on cancer migration or invasion.

11. The method according to claim 8, wherein the cancer includes at least one selected from the group consisting of skin cancer, breast cancer, uterine cancer, colorectal cancer, kidney cancer, liver cancer, lung cancer, ovarian cancer, pancreatic cancer, and gastric cancer.

12. The method according to claim 8, wherein the metabolic disease is selected from the group consisting of diabetes and obesity.

13. The method according to claim 8, wherein the immunological disorder is selected from the group consisting of an autoimmune disease and rheumatoid arthritis.

14. The method according to claim 8, wherein the liver disease is selected from the group consisting of chronic hepatitis, alcoholic cirrhosis, acute liver failure, liver cancer, and fatty liver.

15. A kit for diagnosing cancer, metabolic diseases, immunological disorders, or liver diseases, comprising: a composition comprising at least one selected from the group consisting of.
(a) an extracellular water-soluble domain of delta-like 1 homolog (DLK1), a fragment of the extracellular water-soluble domain of DLK1, a mutant of the extracellular water-soluble domain of DLK1, or a fragment of the mutant as an active ingredient; and
(b) an extracellular water-soluble domain of DLK1 or a fragment thereof, and a DLK1-Fc fusion protein to which an Fc region of a human antibody is linked as an active ingredient.

16. The kit according to claim 15, wherein the kit is an enzyme-linked immunosorbent assay (ELISA) kit or a sandwich ELISA kit.
